(19) United States
(12) Patent Application Publication
(10) Pub. No.: US 2005/0125032 A1
(43) Pub. Date: Jun. 9, 2005

(54) Patent foramen ovale (PFO) closure devices, delivery apparatus and related methods and systems

(76) Inventors: Brian K. Whisenant, Salt Lake City, UT (US); Clark C. Davis, Holladay, UT (US); Daryl Edmiston, Draper, UT (US)

Correspondence Address: Kevin B. Laurence;STOEL RIVES LLP, One Utah Center, 201 South Main Street, Suite 1100, Salt Lake City, UT, 84111, US

(21) Appl. No.: 10/964,311

(22) Filed: Oct. 12, 2004

Related U.S. Application Data
(60) Provisional application No. 60/530,866, filed on Dec. 17, 2003.
(60) Provisional application No. 60/510,263, filed on Oct. 10, 2003.
(60) Provisional application No. 60/510,203, filed on Oct. 10, 2003.

(51) Int. Cl. A61D 1/00; A61B 17/08

(52) U.S. Cl. 606/213; 128/887

(57) ABSTRACT

Devices for closure of a patent foramen ovale, apparatus for delivery of the closure device and methods and systems for closing a patent foramen ovale are disclosed.

### RELATED APPLICATIONS

This utility patent application claims the benefit of Provisional Application Ser. No. 60/530,866 filed on Dec. 17, 2003 and titled COUPLED PATENT FORAMEN OVALE (PFO) CLOSURE APPARATUS AND RELATED METHODS AND SYSTEMS, Provisional Application Ser. No. 60/510,263 filed on Oct. 10, 2003 and titled INTEGRAL PATENT FORAMEN OVALE (PFO) CLOSURE DEVICES AND RELATED METHODS AND SYSTEMS, and Application Ser. No. 60/510,203 filed on Oct. 10, 2003 and titled COUPLEABLE PATENT FORAMEN OVALE (PFO) CLOSURE APPARATUS AND RELATED METHODS AND SYSTEMS. These applications are incorporated herein by reference.

### TECHNICAL FIELD

The present invention relates generally to a patent foramen ovale (“PFO”) in a mammalian heart. More specifically, the present invention relates to apparatus, methods, and systems for closure of a septal defect between the right and left atriums of a patient's heart.

### BRIEF DESCRIPTION OF THE DRAWINGS

Understanding that drawings depict only typical embodiments of the invention and are not therefore to be considered to be limiting of its scope, the invention will be described and explained with specificity and detail through the use of the accompanying drawings. The drawings are listed below.

FIG. 1A is a cross-sectional view of a heart.

FIG. 1B is an enlarged cross-section view of septum primum and the septum secundum and a PFO tunnel between the septum primum and the septum secundum.

FIG. 1C is a perspective view of the septum secundum with the tunnel and the septum primum shown in phantom.

FIG. 2 is a plan view of an embodiment of a PFO closure device 100.

FIG. 3A is an exploded perspective view of PFO closure device 100 and components of a delivery apparatus 200.

FIG. 3B is an assembled side view of PFO closure device 100 and components of delivery apparatus 200 shown in FIG. 3A.

FIG. 4A is a perspective view of PFO closure device 100 while still attached via a threaded detachment tip 210 (not shown in FIG. 4A) to a stem 220. Stem 220 and threaded detachment tip 210 comprises a left atrial anchor (LAA) advancer 230.

FIG. 4B is a cross-sectional view taken at cutting line 4B-4B which shows retainers 140 within anchor connector 150 and threaded detachment tip 210 (not shown in FIG. 4A) while it is still within anchor connector 150 for delivery.

FIG. 4C is a side view of right atrial anchor 170 attached to pivot collar 190 before pivot collar 190 has been pushed fully onto anchor connector 150 and off of stem 220.

FIG. 4D is a top view of right atrial anchor 170 attached to pivot collar 190 before pivot collar 190 has been pushed fully onto anchor connector 150 and off of stem 220.

FIG. 4E is a cross-sectional view of right atrial anchor 170 attached to pivot collar 190 taken on cutting line 4E-4E. FIG. 4E also provides a perspective view of stem 220 as pivot collar 190 is positioned around stem 220 in a configuration which permits pivot collar 190 to be glided on stem 220.

FIG. 4F is an enlarged perspective view of pivot collar 190.

FIG. 4G is a bottom view of pivot collar 190 taken from line 4G-4G.

FIG. 5A is a perspective view of catheter 250 and a cross-sectional view of PFO 50 which depicts an initial step in the method of delivering PFO closure device 100. FIGS. 5B-5P depict subsequent steps.

FIG. 5B is a cross-sectional view of delivery apparatus 200 positioned at PFO 50 to deploy left atrial anchor 130 of closure device 100.

FIG. 5C is perspective view of left atrial anchor 130 as it is being deployed out of catheter 250.

FIG. 5D is a cross-sectional view of left atrial anchor 130 of closure device 100 deployed into left atrium 40.

FIG. 5E is perspective view from within left atrium 40 of left atrial anchor 130 of closure device 100 after it has been deployed into left atrium 40.

FIG. 5F is a cross-sectional view of left atrial anchor 130 of closure device 100 being pulled against septum primum 52 and septum secundum 54 in the left atrium 40.

FIG. 5G is perspective view from within left atrium 140 of left atrial anchor 130 of closure device 100 being pulled against septum primum 52 and septum secundum 54 in the left atrium 40.

FIG. 5H is a cross-sectional view of right atrial anchor 170 of closure device 100 being deployed in right atrium 30.

FIG. 5I is perspective view from within right atrium 30 of right atrial anchor 170 after deployment and ready for clockwise rotation by right atrial anchor (RAA) advancer 270.

FIG. 5J is a cross-sectional view of right atrial anchor 170 of closure device 100 being deployed in right atrium 30.

FIG. 5K is perspective view from within right atrium 30 of right atrial anchor 170 positioned under the overhang of septum secundum 54.

FIG. 5L is a cross-sectional view of right atrial anchor 170 being advanced on anchor connector 150 toward left atrial anchor 130.

FIG. 5M is perspective view from within right atrium 30 of right atrial anchor 170 as positioned on anchor connector 150 by right atrial anchor (RAA) advancer 270.

FIG. 5N is a cross-sectional view of closure device 100 and delivery apparatus 200 after removal of left atrial anchor (LAA) advancer 230.

FIG. 5O is perspective view from within right atrium 30 of closure device 100 and right atrial anchor (RAA) advancer 270 of delivery apparatus 200 after removal of left atrial anchor (LAA) advancer 230.

FIG. 5N is a cross-sectional view of closure device 100 and delivery apparatus 200 after removal of right atrial anchor (LAA) advancer 270 and catheter 250.

FIG. 5P is perspective view from within right atrium 30 of closure device 100 positioned in PFO 50 after removal of delivery apparatus 200.

FIG. 6A is a plan view of an embodiment of a PFO closure device 100′.

FIG. 6B is an assembled side view of PFO closure device 100′ and components of delivery apparatus 200′.

FIG. 6C is an exploded perspective view of right atrial anchor 170′ and right atrial anchor (RAA) retainer 190′, also referred to herein as a pivot collar 190′.

FIGS. 6D is a cross-sectional view taken along cutting line 6D-6D which depicts pivot collar 190′ as positioned in right atrial anchor 170′.

FIG. 6E is a perspective view of closure device 100′ (with right atrial anchor 170′ shown in a cross-sectional view) and components of delivery apparatus 200 including coupler 290′.

FIG. 6F is a perspective view of closure device 100′ (with right atrial anchor 170′ shown in a cross-sectional view) and coupler 290′ engaging pivot members 190′ of pivot collar 190′.

FIGS. 6G is a cross-sectional view taken along cutting line 6G-6G which depicts coupler 290′ engaging pivot members 194′ of pivot collar 190′.

FIG. 7A is a perspective view depicting another embodiment of a right atrial anchor at 170a.

FIG. 7B is a perspective view depicting another embodiment of a right atrial anchor at 170b.

FIG. 7C is a perspective view depicting another embodiment of a right atrial anchor at 170c.

FIG. 7D is a plan view depicting another embodiment of a right atrial anchor at 170d.

FIG. 7E is a side view of the embodiment of right atrial anchor 170d shown in FIG. 7E.

FIG. 8A is perspective view from within right atrium 30 of closure device 100 positioned in PFO 50 with both ends of right atrial anchor 170 positioned within pockets 59a and 59p.

FIG. 8B is perspective view from within right atrium 30 of closure device 100 positioned in PFO 50 with one end of right atrial anchor 170 positioned within pocket 59p.

FIG. 8C is perspective view from within right atrium 30 of closure device 100 positioned in PFO 50 with both ends 171 of right atrial anchor 170a positioned within pockets 59a and 59p.

FIG. 8D is perspective view from within right atrium 30 of closure device 100 positioned in PFO 50 with one end 171 of right atrial anchor 170a positioned within pocket 59p.

FIG. 9 is plan and cross-sectional view of another embodiment of a left atrial anchor as identified at 130′.

FIG. 10 is perspective view of another embodiment of a left atrial anchor as identified at 130″.

FIG. 11 is cross-sectional view of another embodiment of a left atrial anchor as identified at 130′″.

FIG. 12A is a cross-sectional view of another embodiment of a closure device 100i a having a left atrial anchor 130a and another embodiment of a delivery apparatus 200″ having a left atrial anchor (LAA) advancer 230″.

FIG. 12B provides a perspective view of left atrial anchor 130a as depicted in FIG. 12A during deployment and a cross-section view of catheter 250″ to show right atrial anchor (LM) advancer 270″.

FIG. 12C provides a perspective view of left atrial anchor 130a as compressed in a left atrium and right atrial anchor 170″ as positioned in the right atrium by right atrial anchor (LAA) advancer 270″.

FIG. 13A is a plan view of left atrial anchor 130a shown in FIGS. 12A-12C.

FIG. 13B is a plan view of another embodiment of a left atrial anchor as identified at 130b.

FIG. 13C is a plan view of another embodiment of a left atrial anchor as identified at 130c.

FIG. 13D is a plan view of another embodiment of a left atrial anchor as identified at 130d.

FIG. 13E is a plan view of another embodiment of a left atrial anchor as identified at 130e.

FIG. 13F is a plan view of another embodiment of a left atrial anchor as identified at 130f as combined with links 122f.

FIG. 14A is an enlarged cross-sectional view of the joint identified at 135a.

FIG. 14B is an enlarged cross-sectional view of the joint identified at 135b.

FIG. 14C is an enlarged cross-sectional view of the joint identified at 135c.

FIG. 14D is a side view of left atrial anchor 130d.

FIG. 15A is a plan view of web 122 for combination with left atrial anchor members of left atrial anchor 130e.

FIG. 15B is a plan view of web 122′ for combination with left atrial anchor members of left atrial anchor 130e.

FIG. 15C is a side view of left atrial anchor 130f and anchor connector 150f.

### INDEX OF ELEMENTS IDENTIFIED IN THE DRAWINGS

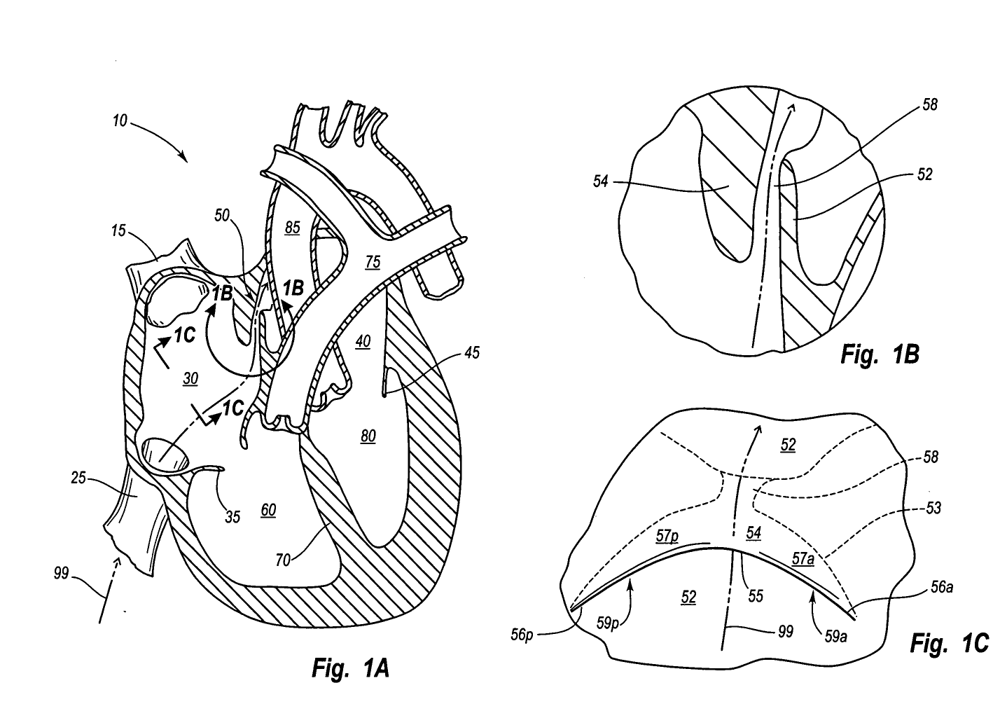

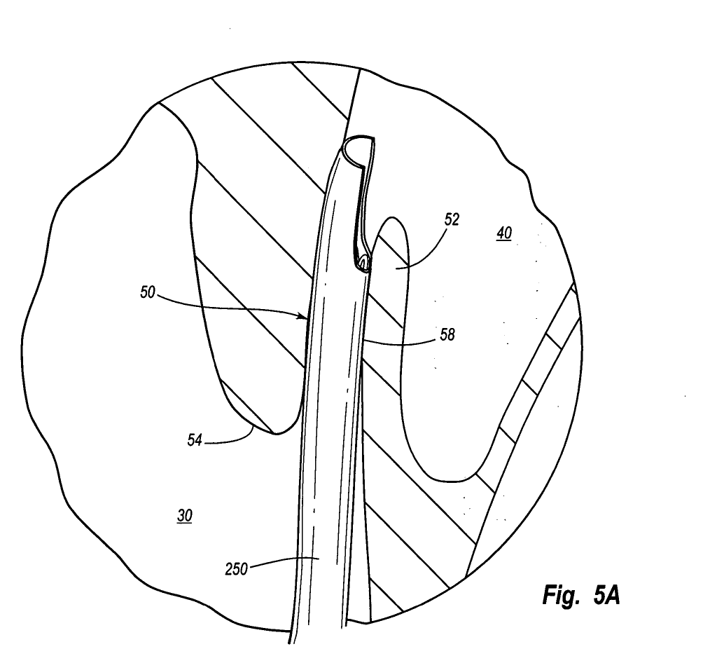

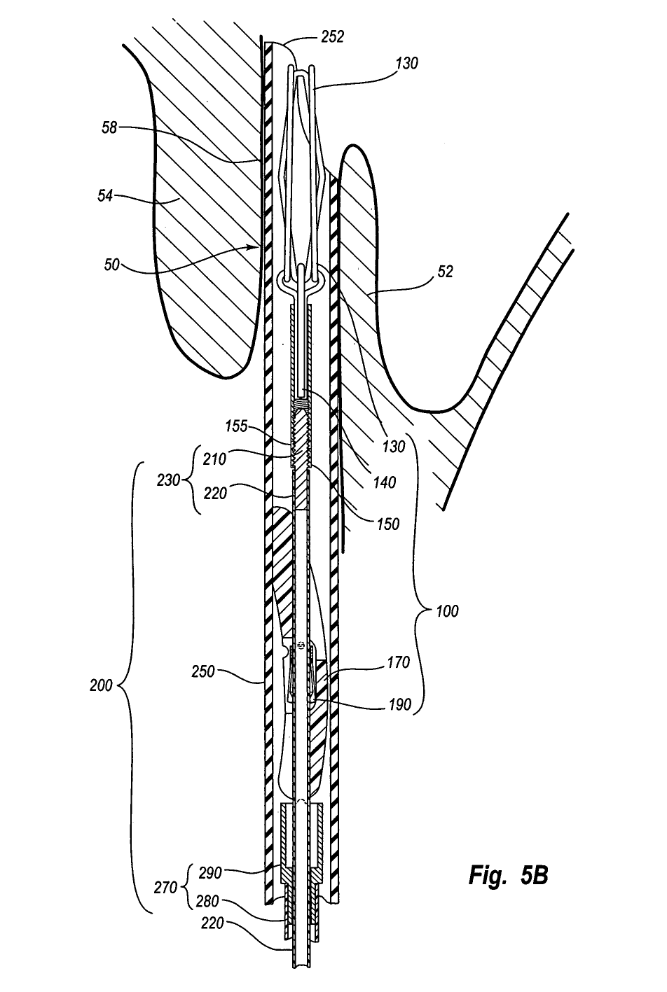

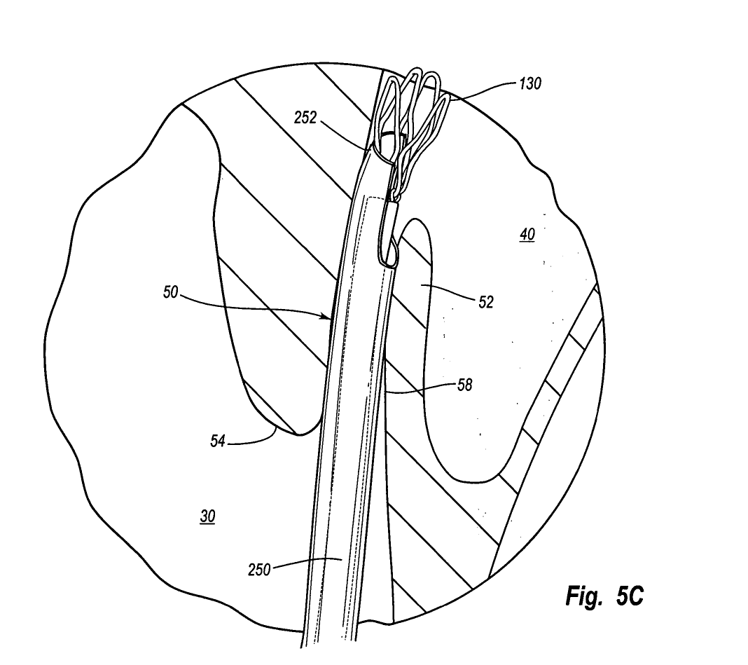

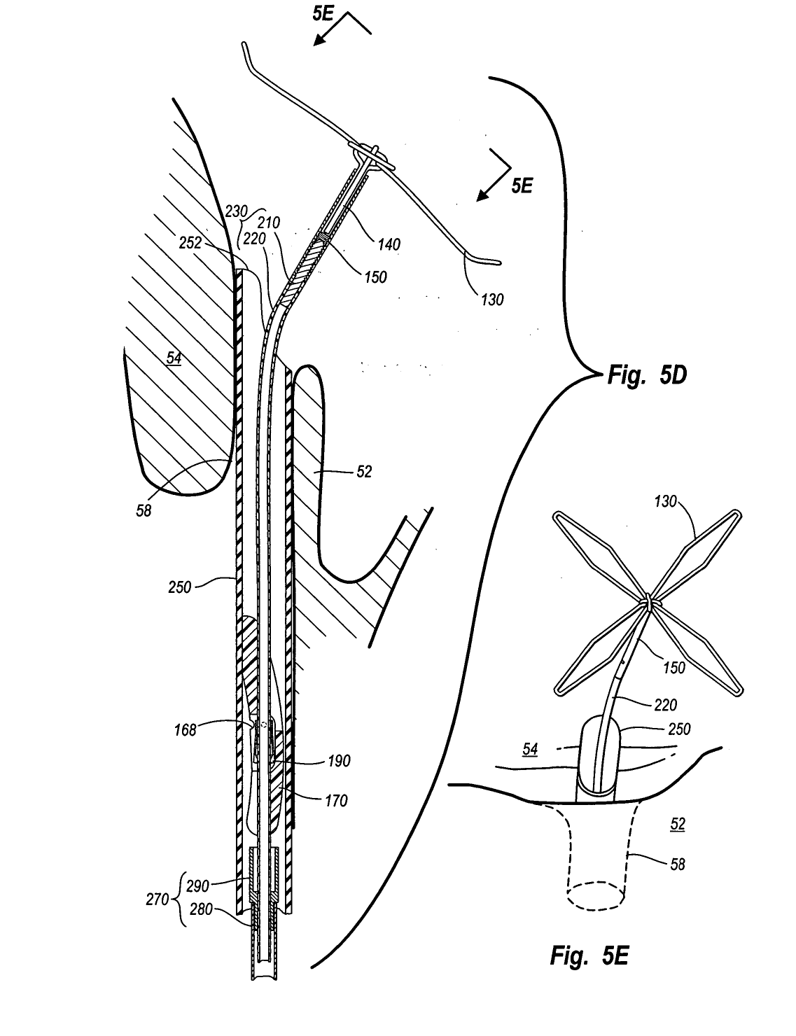

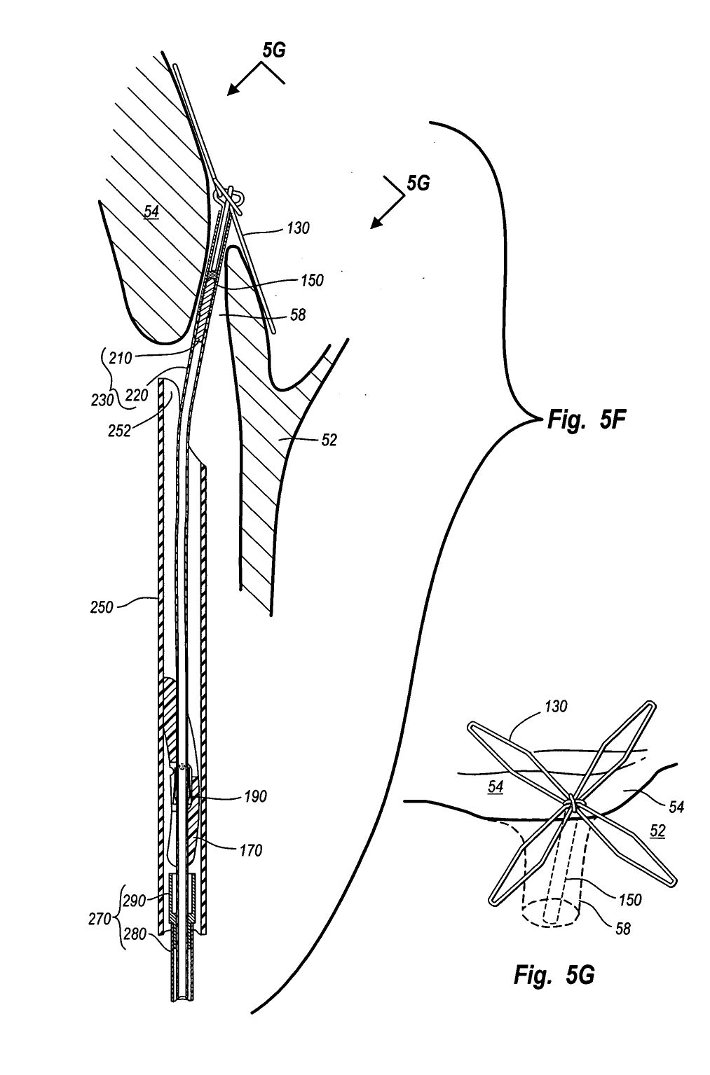

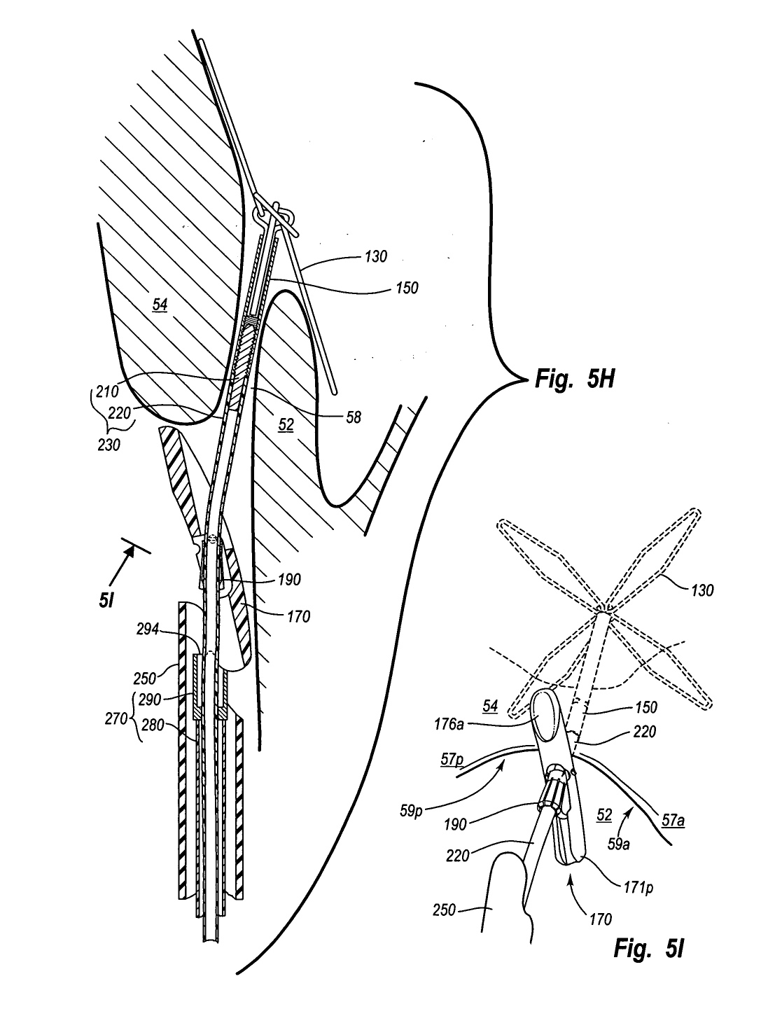

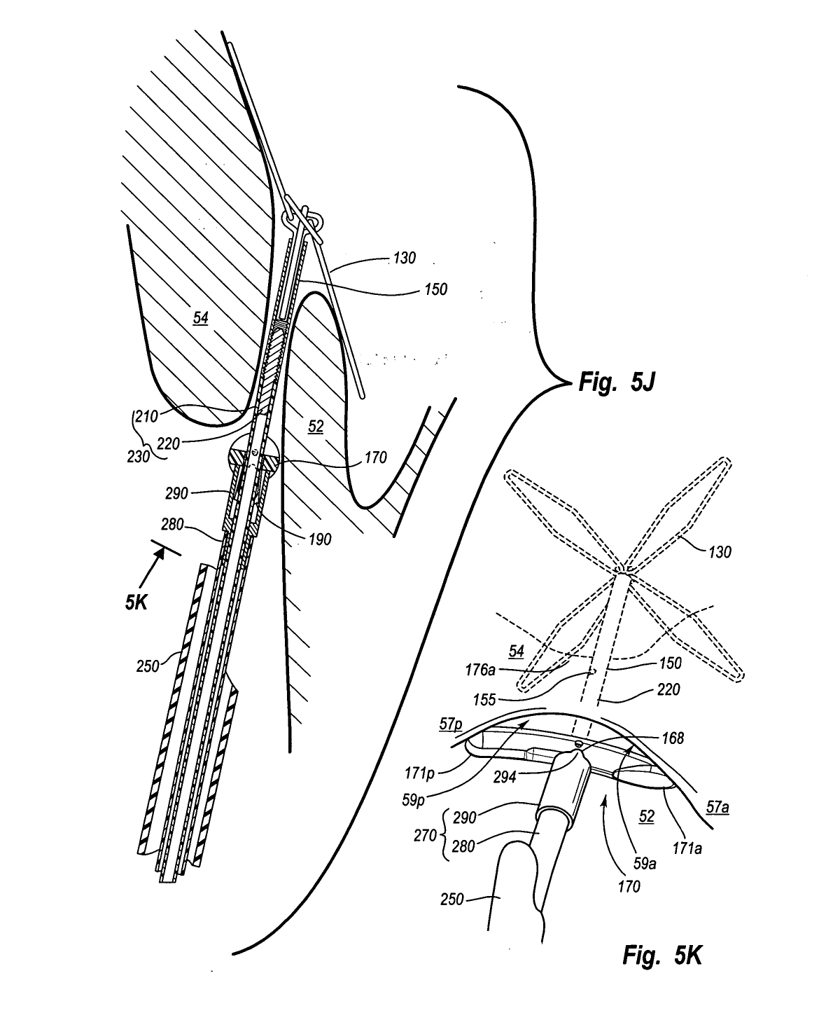

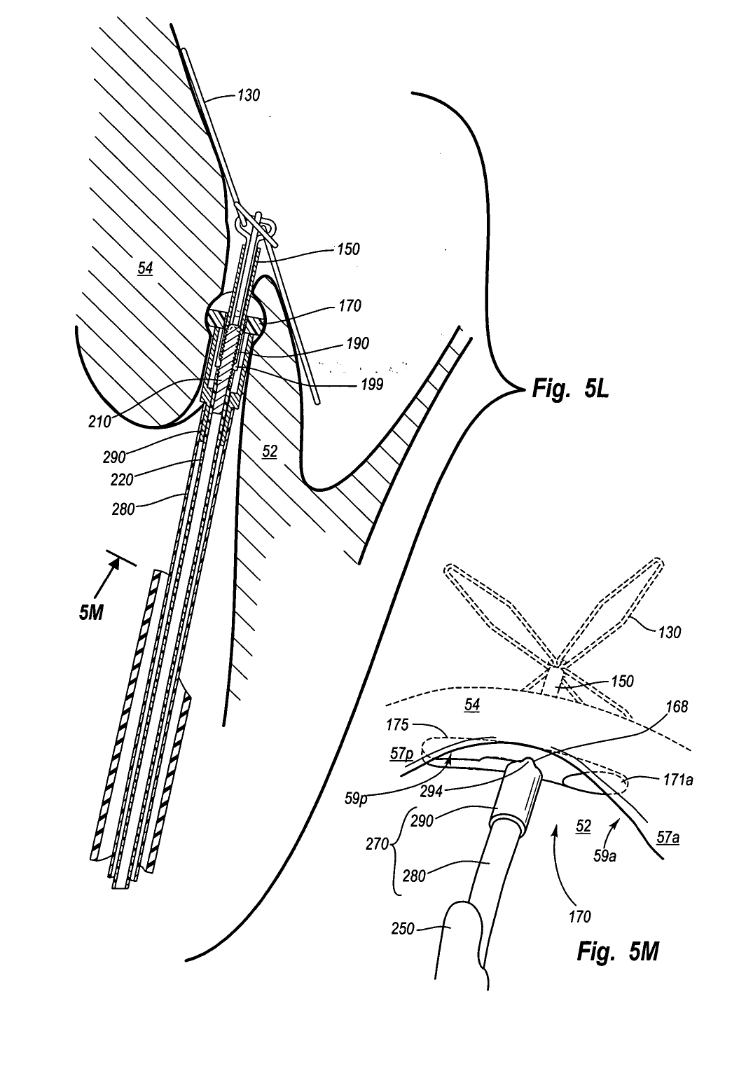

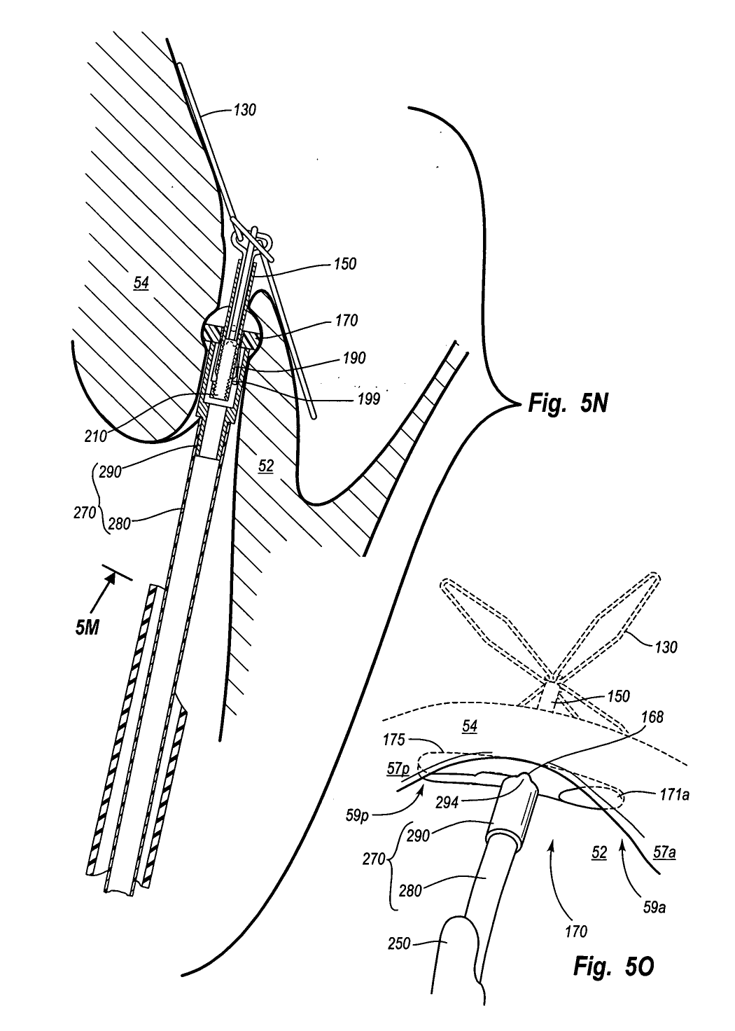

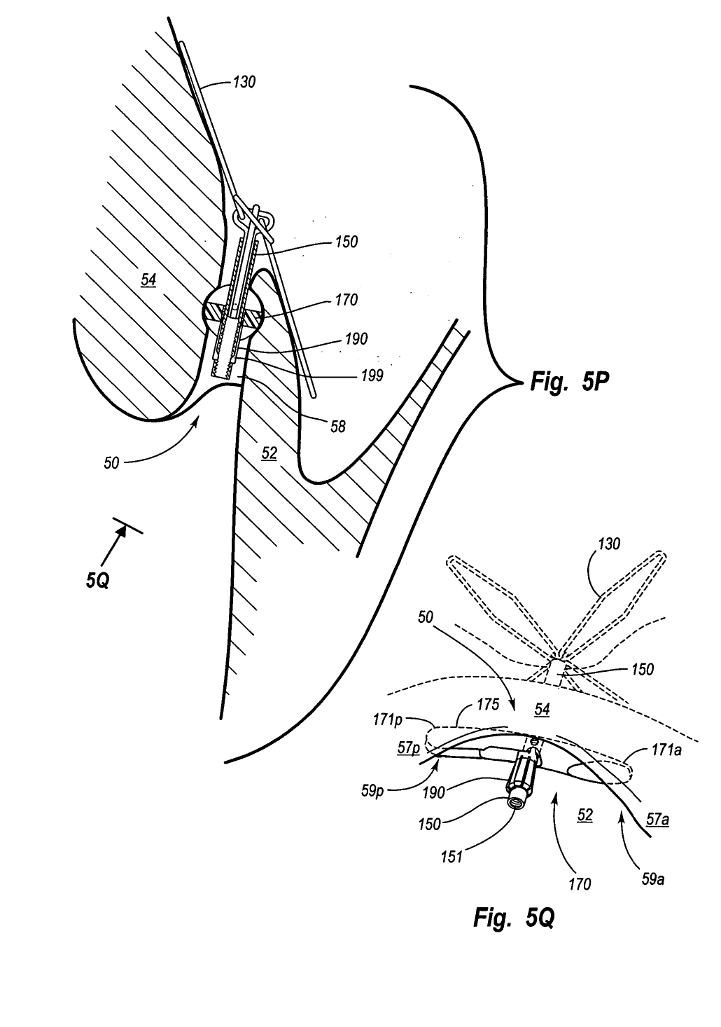

Elements of the heart 10 are shown in FIGS. 1A-1C. Some of these elements are also shown in one or more of or are discussed with reference FIGS. 5A-5Q, 8A-8D, and 11. These elements include:
15 superior vena cava
25 inferior vena cava
30 right atrium
35 tricuspid valve
40 left atrium
45 bicuspid valve
50 PFO
52 septum primum
53 superior aspect
54 septum secundum
56a anterior merger point
56p posterior merger point
57a anterior portion
57p posterior portion
58 tunnel
59a anterior pocket
59p posterior pocket
60 right ventricle
70 interventricular septum
75 pulmonary veins
80 left ventricle
85 aorta
99 delivery path

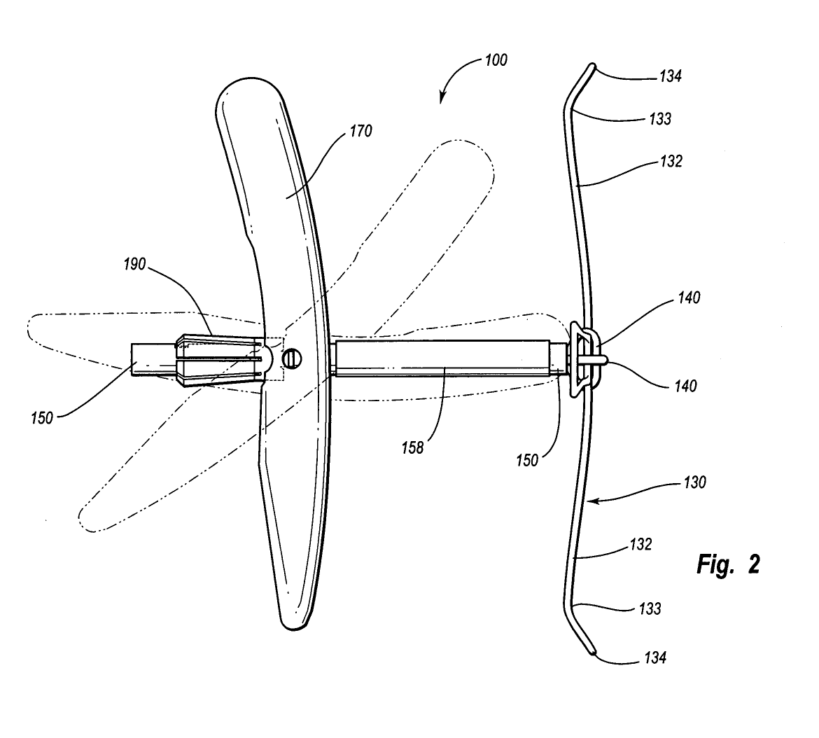

The elements listed below are components of patent foramen ovale (PFO) closure device 100 or other embodiments including 100′, 100″, 100′″ and 100a. Note that all features or subcomponents of components even those which relate only to a particular embodiment are listed below without reference to the particular embodiment. For example, left atrial anchors 130a-f and right atrial anchors 170′ and 170a-d include certain features and subcomponents which are unique to the particular embodiment, however, they are generically included in this list and are not individually listed. The following elements are shown in one or more of or are discussed with reference to FIGS. 2, 3A-3B, 4A-4G, 5B-5Q, 6A-6G, 7A-7C, 8A-8D, 9, 10, 11, 12A-12C, 13A-13F, 5A-15C. These elements include:
120 mesh
122 web
123 arm link
124 perimeter link
125 inset link
130 left atrial anchor
132 anchor member
133 flex point
134 tips
135 joints (referenced to LAA 130a-c)
138 first center feature (referenced to LM 130a and LAA 130d)
139 second center feature (referenced to LAA 130a and LAA 130d)
140 left atrial anchor retainer
150 anchor connector
151 threads
152 stop
153 end (referenced to anchor connector 150a)
155 retention holes
157 right atrial anchor (RAA) end of anchor connector 150
158 coating
162 non-resorbable components (referenced to RAA 170b-c)
164 resorbable components (referenced to RAA 170b-c)
166 notches (referenced to RAA 170b-c)
168 torque groove
170 right atrial anchor
171a anterior end of right atrial anchor 170
171p posterior end of right atrial anchor 170
172a stem groove of anterior end 171a
172p stem groove of posterior end 171p
173a stem chamber of anterior end 171a
173p stem chamber of posterior end 171p
174 hole
175 top surface or contact surface
176a flat portion
176p rounded portion
177 concave portion
178 pivot groove
179 pivot chamber
180 loop or flex point or region
184 opening in right atrial anchor
190 right atrial anchor (RAA) retainer, pivot collar or locking arm
191 groove
192 band (referenced with pivot collar 190′)
194 pivot members
195 ferrule (referenced with pivot collar 190′)
196 body portion
199 retention pawls

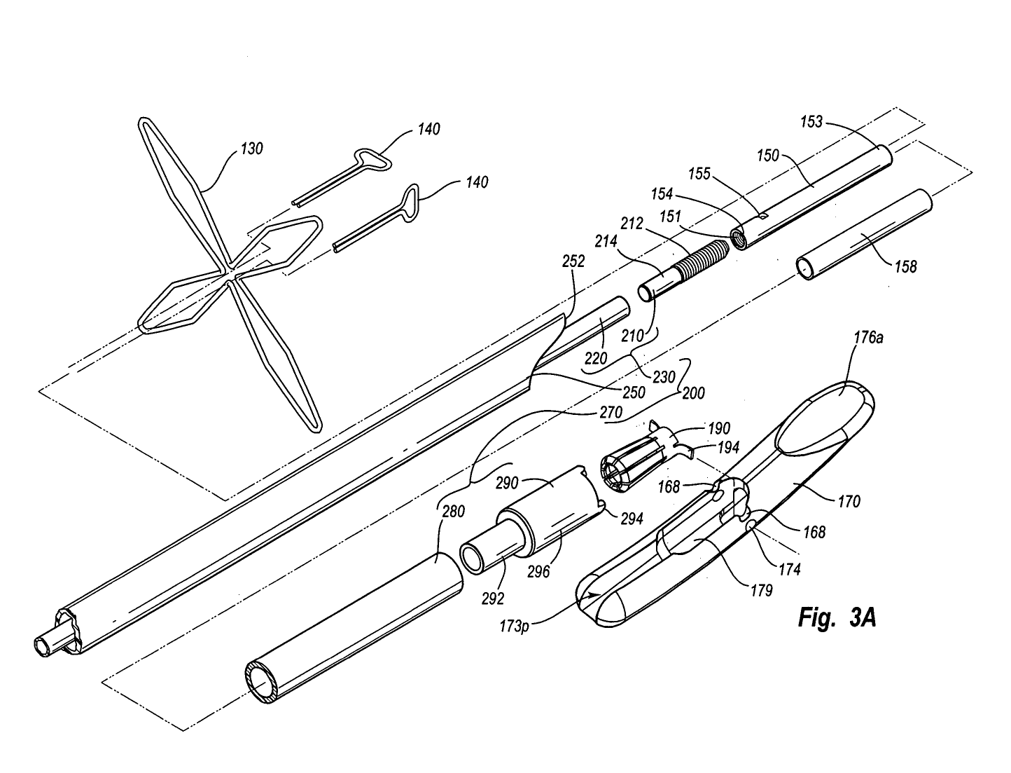

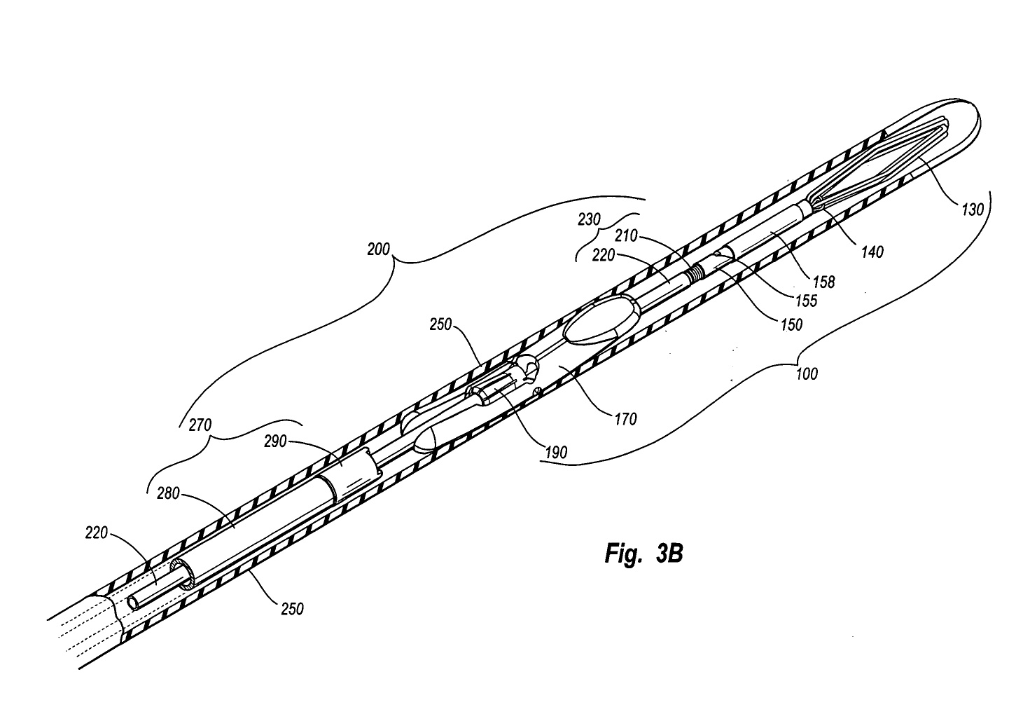

The elements listed below are components of delivery apparatus 200, 200′, 200″ or other embodiments. The following elements are shown in one or more of or discussed with reference to FIGS. 3A-3B, 4A, 4E, 5A-50, 6B, 6E-6G, and 12A including:
210 threaded detachment tip
212 threads
220 stem
230 left atrial anchor (LM) advancer
250 catheter
270 right atrial anchor (RAA) advancer
280 stem
290 coupler
294 torque feature

### DETAILED DESCRIPTION OF PREFERRED EMBODIMENTS

FIGS. 1A-1C depict various views of a heart. Heart 10 is shown in a cross-section view in FIG. 1A. In a normal heart, the right atrium 30 receives systemic venous blood from the superior vena cava 15 and the inferior vena cava 25 and then delivers the blood via the tricuspid valve 35 to the right ventricle 60. However, in heart 10, there is a septal defect between right atrium 30 and left atrium 40 of a patient's heart which is referred to as a patent foramen ovale (“PFO”). The PFO, which is an open flap on the septum between the heart's right and left atria, is generally identified at 50. In a normal heart, left atrium 40 receives oxygenated blood from the lungs 40 via pulmonary veins 75 and then delivers the blood to the left ventricle 80 via the bicuspid valve 45. However, in heart 10 some systemic venous blood also passes from right atrium 30 through PFO 50, mixes with the oxygenated blood in left atrium 40 and then is routed to the body from left ventricle 80 via aorta 85.

During fetal development of the heart, the interventricular septum 70 divides right ventricle 60 and left ventricle 80. In contrast, the atrium is only partially partitioned into right and left chambers during normal fetal development as there is a foramen ovale. When the septum primum 52 incompletely fuses with the septum secundum 54 of the atrial wall, the result is a PFO, such as the PFO 50 shown in FIGS. 1A-1C, or an atrial septal defect referred to as an ASD.

FIG. 1C provides a view of the crescent-shaped, overhanging configuration of the typical septum secundum 54 from within right atrium 30. Septum secundum 54 is defined by its inferior aspect 55, corresponding with the solid line in FIG. 1C, and its superior aspect 53, which is its attachment location to septum primum 52 as represented by the phantom line. Septum secundum 54 and septum primum 52 blend together at the ends of septum secundum 54; these anterior and posterior ends are referred to herein as “merger points” and are respectively identified at 56a and 56p. The length of the overhang of septum secundum 54, the distance between superior aspect 53 and inferior aspect 55, increases towards the center portion of the septum secundum as shown. A tunnel 58 is defined by portions of septum primum 52 and septum secundum 54 between the merger points 56a and 56p which have failed to fuse. The tunnel is often at the apex of the septum secundum as shown. When viewed within right atrium 30, the portion of septum secundum 54 to the left of tunnel 58, which is referred to herein as the posterior portion 57p of the septum secundum, is longer than the portion of the septum secundum 54 to the right of tunnel 58, which is referred to herein as the anterior portion 57a of the septum secundum. In addition to being typically longer, the left portion also typically has a more gradual taper than the right portion, as shown. The area defined by the overhang of the anterior portion 57a of septum secundum 54 and the septum primum 52 and extending from the anterior merger point 56a toward tunnel 58 is an anterior pocket 59a. Similarly, the area defined by the overhang of the posterior portion 57p of septum secundum 54 and the septum primum 52 and extending from the posterior merger point 56p toward tunnel 58 is a posterior pocket 59p.

The invention described hereinafter relates to a closure device, a delivery apparatus, methods, and systems for closure of a PFO. FIG. 2 depicts one embodiment of a closure device at 100. FIGS. 3A-3B depict closure device 100 and an embodiment of a delivery apparatus 200.

Closure device 100 comprises a left atrial anchor 130 and a right atrial anchor 170. In the embodiment of the closure device shown in FIG. 2, left atrial anchor 130 and right atrial anchor 170 are coupled together via an anchor connector 150. Left atrial anchor 130 is secured to anchor connector 150 via two left atrial anchor (LAA) retainers 140. While the components described above are separate, several of these components may alternatively be integral. For example, in another embodiment, left atrial anchor 130, retainer 140 and/or anchor coupler 150 may be integral. Right atrial anchor 170 is secured to anchor connector 150 by a right atrial anchor (RAA) retainer. The embodiment of right atrial anchor (RAA) retainer identified at 190 is referred to herein as a pivot collar.

Anchor connector may alternatively be coated with a coating 158 as may left atrial anchor 130, right atrial anchor 170 and any other component of closure device 100 to facilitate closure of PFO 50. Such coatings may be applied to promote occlusion of tunnel 58 and endothelial growth while minimizing thrombosis and embolization. For example, a coating of bioresorbable polymers may be applied which facilitates closure of tunnel 58. Examples of suitable bioresorbable polymers include polycaprolactones, polyorthoesters, polylactide, polyglycolide and copolymers of these polymers. An example of a suitable copolymer is polylactide and polyglycolide. In addition to polymers, drug eluting compositions, proteins and growth factors may also be applied as coatings. Examples of suitable proteins and growth factors include elastin, fibronectin, collagen, laminin, basic fibroblast growth factor, platelet-derived growth factor. The coating may be cellular or foamed or may be more dense as needed. The material used for the coating may depend on the particular component of closure device 100 being coated. For example, elastin is useful for coating left atrial anchor 130 and right atrial anchors as it is not aggressive for tissue growth. Anchor connector 150 may be wrapped with a foam material, fuzzy bioresorbable thread or any other material which assists in facilitating the closure of tunnel 58.

By coating components of closure device 100 such as left atrial anchor 130, anchor connector 150 and right atrial connector 170, tissue growth can be promoted at the points of contact of each of these three components in three regions or planes. Note that the components of the closure device may also be formed entirely from the materials listed above for coatings.

FIG. 3A provides an exploded perspective view of closure device 100 and some components of delivery apparatus 200. FIG. 3B provides a cross-sectional view of the same components. Components of delivery apparatus 200 shown in FIGS. 3A-3B include a left atrial anchor (LAA) advancer 230 for advancing left atrial anchor 130, a right atrial anchor (RAA) advancer 270 for advancing right atrial anchor 170 and catheter 250. Left atrial anchor (LAA) advancer 230 comprises a stem 220 which is fixedly or integrally coupled to a threaded detachment tip 210. Right atrial anchor (RAA) advancer 270 comprises a stem 280 and a coupler 290. Left atrial anchor (LAA) advancer 230 pass through right atrial anchor (RAA) advancer 270.

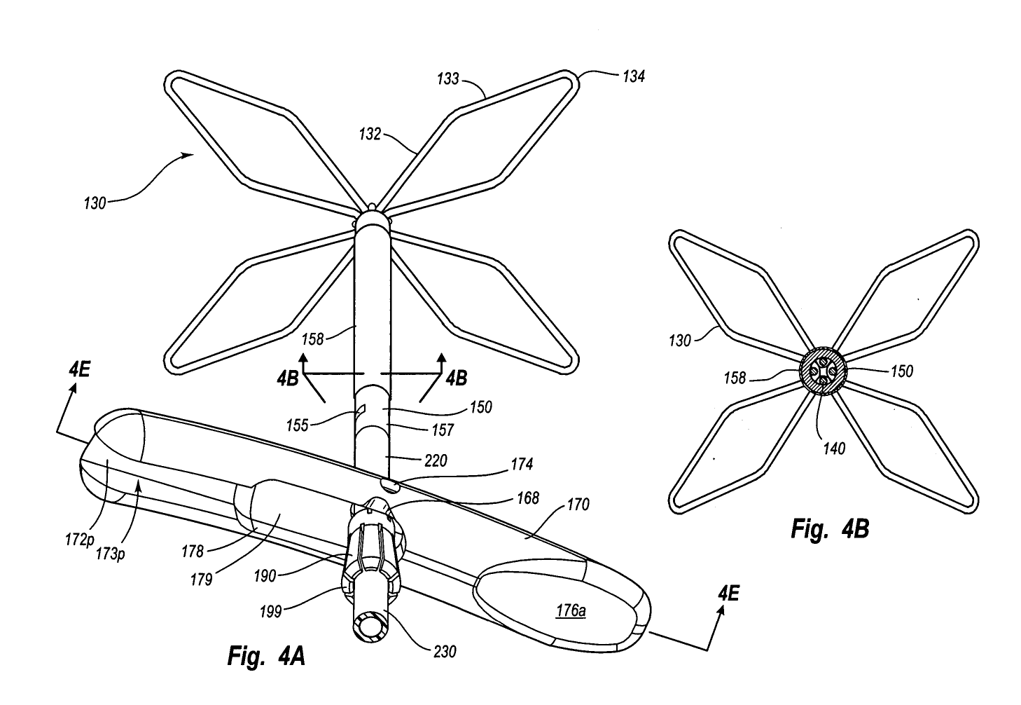

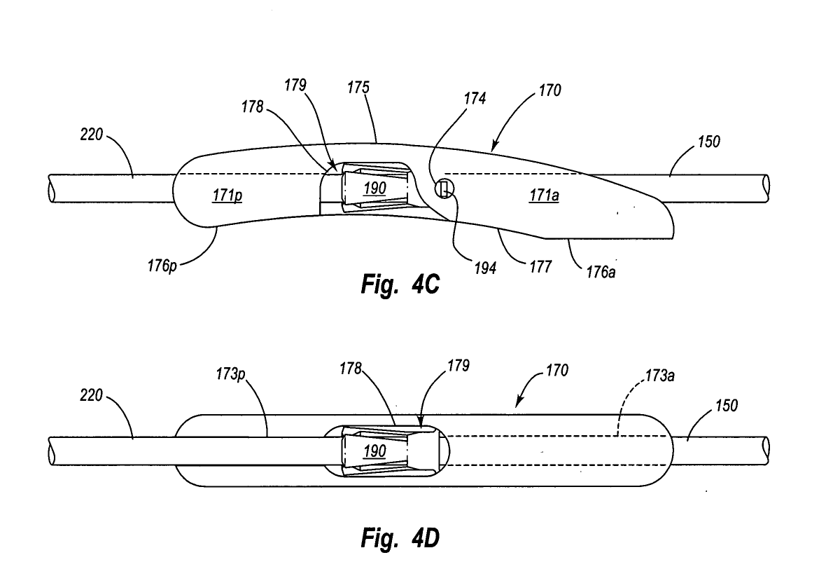

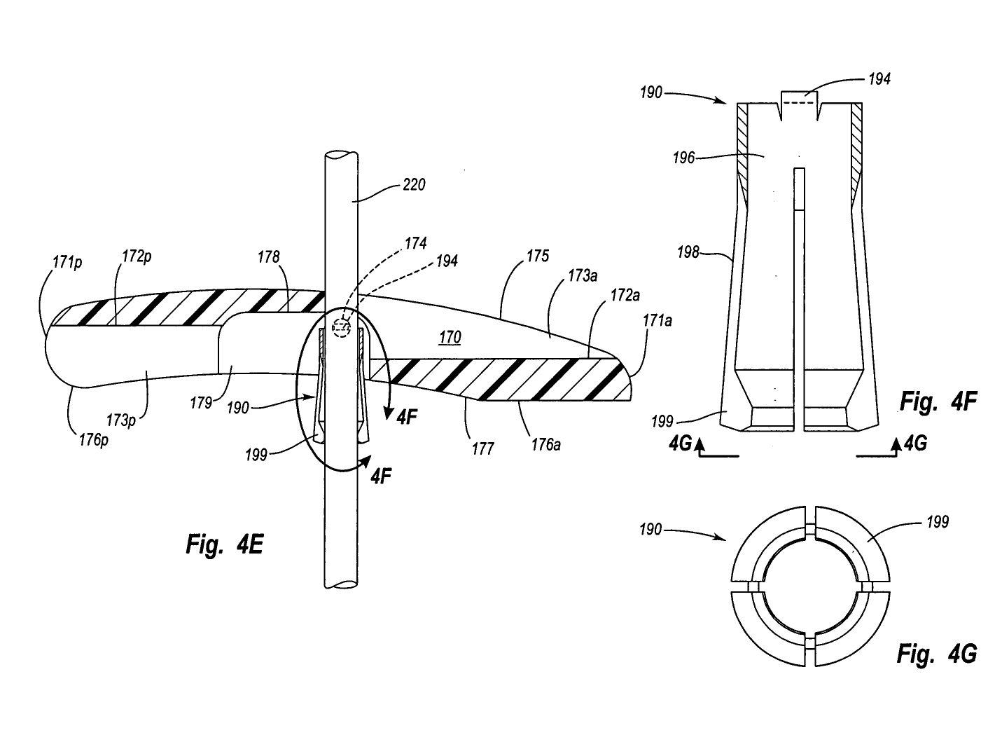

FIGS. 4A-4G show additional features of closure device 100 particularly, right atrial anchor 170. The functions of these features are best understood with reference to FIGS. 5A-5P.

FIG. 4A provides a perspective view of closure device 100 with anchor connector 150 still attached to stem 220 of left atrial anchor (LAA) advancer 230. Right atrial anchor 170 has not yet been advanced into its final position on the right atrial anchor (RAA) end 157 of anchor connector 150. Hole 155 in end 157 of anchor connector 150 are shown in FIG. 4A ready to receive retention pawls 199 of pivot collar 190, which is more generally referred to as a right atrial anchor (RAA) retainer.

FIG. 4B provides a cross-section view of anchor connector 150 taken at cutting line 4B-4B. FIG. 4B shows retainers 140 within anchor connector 150 and a coating 158 on anchor connector 150.

FIG. 4C is a side view of right atrial anchor 170 attached to pivot collar 190 before pivot collar 190 has been pushed fully onto anchor connector 150 and off of stem 220. FIG. 4D is a top view of right atrial anchor 170 attached to pivot collar 190; in the same position as is shown in FIG. 4C. FIG. 4E provides a cross-sectional view of right atrial anchor 170 taken on cutting line 4E-4E, right atrial anchor 170 is in the same position as FIGS. 4C-4D on stem 220 after being rotated. FIG. 4E also provides a perspective view of stem 220 as pivot collar 190 is positioned around stem 220 in a configuration which permits pivot collar 190 to be glided on stem 220.

Right atrial anchor 170 has two opposing ends which are respectively adapted to be positioned in anterior pocket 59a and posterior pocket 59p. The opposing end identified at 171a may be placed in anterior pocket 59a or adjacent to the anterior portion 57a of septum secundum 54. Similarly, the opposing end of right atrial anchor 170 identified at 171p may be placed in posterior pocket 59p or adjacent to the posterior anterior portion 57p. Right atrial anchor is relatively symmetrical so that end 171p or end 171a can be positioned in either posterior pocket 59p or anterior pocket 59a. Accordingly, the use of the designations “a” and “p” to designate an eventual position with either an anterior or posterior orientation does not indicate that either end 171a or end 171p must be positioned to have respective anterior and posterior orientations.

To permit right atrial anchor 170 to be easily moved within a catheter, right atrial anchor 170 has three chambers which are adapted to fit around pivot collar 190, anchor connector 150 and stem 220. A stem groove is formed in the two opposing ends of right atrial anchor 170 as identified at 172a and 172p which each respectively defined a stem chamber 173a and 173p. Pivot collar 190 has pivot members 194 which are received within holes 174 to permit right atrial anchor to pivot with respect to pivot collar 190. Right atrial anchor 170 has a pivot groove 178 which defines a pivot chamber 179. In this embodiment, the chambers described above allow relatively concentric movement of right atrial anchor 170 with respect to catheter 250 shown in FIG. 5B, anchor connector 150 and stem 220.

Right atrial anchor 170 has a top surface 175 which has a convex shape. The convex shape of top surface 175 permits optimal anatomical conformance with the shape of septum secundum 54. Note that the shape of surface 175 on either side of pivot groove 178 is essentially the same to permit right atrial anchor to oriented with ends 171a and 171p respectively positioned adjacent to portions 57p and 57a or vice versa. Right atrial anchor has a flat portion 176a opposite a rounded portion 176p at its bottom surface. Flat portion 176a provides for an optimal fit within catheter 250. The bottom surface includes a concave portion 177 between flat portion 176a and rounded portion 176p. Concave portion 177 is shaped to minimize the size of right atrial anchor 170.

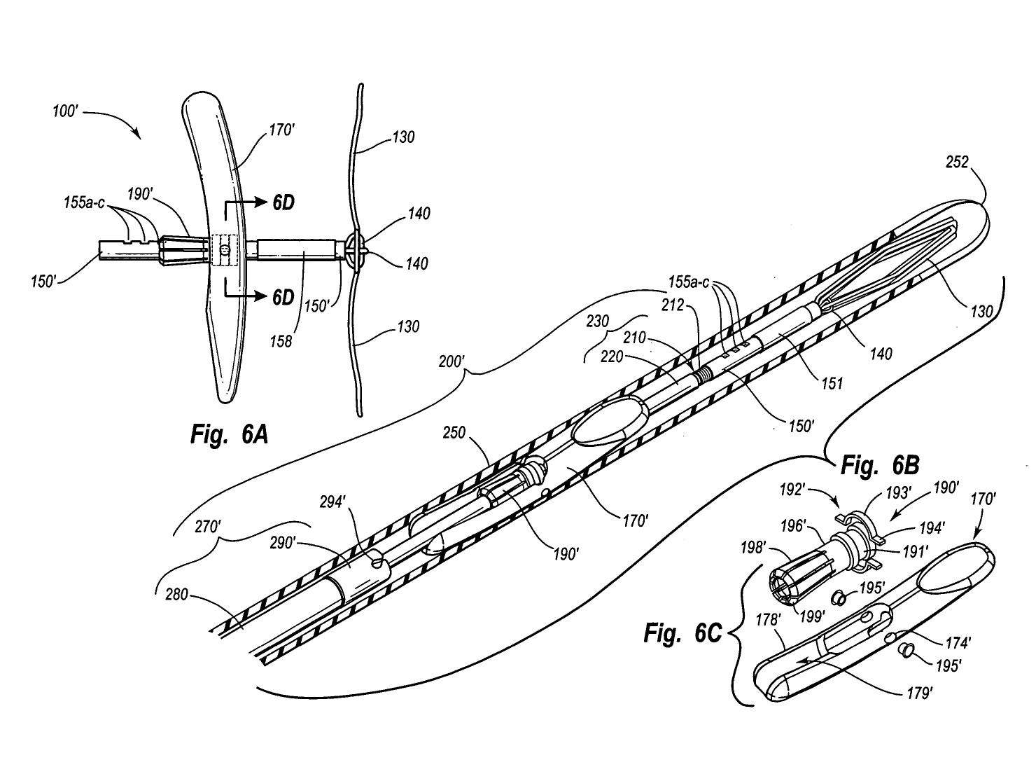

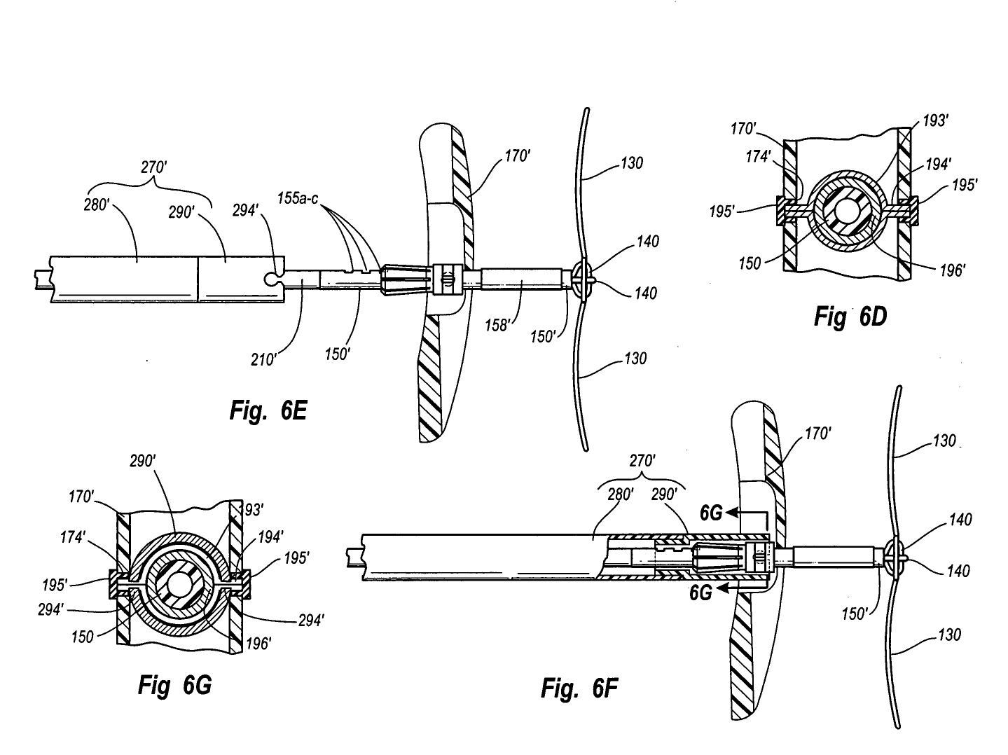

Right atrial anchor 170 has a torque groove 168 which is adapted to fit in a mated with a complimentary torque feature 194. The interaction of torque groove 168 and torque feature 194 to rotate and move right atrial anchor 170 is described below with reference to FIGS. 5I-5O. Another embodiment of a torque feature for rotation and movement of a right atrial anchor is described below with reference to FIGS. 6A-6G.

Details of pivot collar 190 can be easily seen in the enlarged cross-sectional view of FIG. 4F and the view of pivot collar provided by FIG. 4G which is taken along line 4G-4G. Note that another embodiment of a right atrial anchor (RAA) retainer identified at 190′ is discussed below in relation to FIG. 6C. As mentioned above, pivot collar 190 has pivot members 194 which are received within holes 174 to permit right atrial anchor to pivot with respect to pivot collar 190. Pivot members 194 extend from body portion 196. A plurality of arms 198 extend from body portion 196. Each arm 198 has a retention pawl 199. As mentioned above, retention pawls 199 enter retention hole 155 of anchor connector 150 to secure pivot collar 190 to anchor connector 150.

FIGS. 5A-5P depict one method for delivering closure device 100 to PFO 50 via delivery apparatus 200 and deploying closure device 100. Steps involved in recapturing closure device 100 are shown in FIGS. 6A-6G.

Catheter 250 is introduced to PFO 50 via delivery path 99 which is identified in FIGS. 1A-1C. Catheter 250 is a long somewhat flexible catheter or sheath introduced into a vein such as the femoral vein and routed up to the right atrium of a patient's heart. The catheter may be tracked over a guide wire that has been advanced into the heart by a known methodology. After catheter 250 is introduced into the heart via inferior vena cava 25, catheter 250 is positioned at right atrium 30 in front of the interatrial communication or PFO, and then through tunnel 58. Once the distal end of 252 of catheter 250 is positioned at the end of tunnel 58 as shown in FIGS. 5A-5B or extends beyond tunnel 58, left atrial anchor 130 is deployed as shown in FIG. 5D.

FIG. 5B provides a cross-sectional view of closure device 100 and delivery apparatus 200 just before left atrial anchor 130 is pushed out of catheter 250 and deployed into left atrium 40. As indicated above, left atrial anchor (LAA) advancer 230, more particularly stem 220 and threaded detachment tip 210, move within right atrial anchor (RAA) advancer 270, more particularly stem 280 and coupler 290, to advance left atrial anchor 130 within catheter 250.

FIG. 5C depicts left atrial anchor 130 just before deployment and FIG. 5D depicts left atrial anchor 130 after deployment. As provided below, the left atrial anchor may have many different configurations which permit it to fit within the catheter, either by being rotatably or pivotally aligned with the axis of the catheter or by being sufficiently flexible to fit within the catheter in a compressed and/or flexed state. The state in which a left atrial anchor is within the catheter will be referred to herein as a delivery configuration. The state in which an anchor is outside of the catheter and has been pivoted, rotated, flexed, expanded, or otherwise put in position to be placed at the PFO site will be referred to herein as a deployed configuration.

Depending on the particular embodiment of left atrial anchor, in deploying the left atrial anchor from the catheter, it will be expanded, pivoted, or rotated to extend once out of the catheter. The embodiment of the left atrial anchor depicted in FIG. 5D expands and pivots from the delivery configuration to a deployed configuration. Left atrial anchor 130 may be formed from any suitable material such as coiled metal, coiled polymer or a solid core of metal or plastic wrapped with metal or polymer coil. For example, left atrial anchor may be formed from super elastic nickel/titanium or nitinol. It may have a single strand core or a core with multiple strands. The core may be wrapped with metal wire formed from a dense biocompatible metal such as platinum, platinum/tungsten alloy, platinum/iridium alloy, or platinum/iridium/rhodium alloy to increase the radio-opacity of the left atrial anchor. Utilizing a multiple strand core permits the left atrial anchor to have lower bending stiffness and better memory compared with a left atrial anchor formed with a single strand having approximately the same cross-sectional area as the multiple strands.

FIG. 5E shows the appearance of left atrial anchor 130 from within left atrium 40 once left atrial anchor 130 has been deployed. Catheter 250 is shown extending beyond tunnel 58.

FIGS. 5F-5G show left atrial anchor being pulled proximally and positioned proximate to the PFO. For embodiments such as left atrial anchor 130, the left atrial anchor pivots at or near its center. This pivoting motion permits the left atrial anchor to conform to the surfaces of the septum secundum and the septum primum. Once left atrial anchor 130 is pivoted at an angle with respect to the axis of the anchor connector 150, left atrial anchor 130 is pulled flush against septum secundum 54 and septum primum 52. As explained above, each anchor member 132 is angled. More particularly, each anchor member 132 is bowed such that there is a flex point 133 along its length. Pulling left atrial anchor 130 flush against septum secundum 54 and septum primum 52 flattens anchor members 132 of left atrial anchor 130 and enables left atrial anchor 130 to push against septum secundum 54 and septum primum 52 when closure device 100 is finally positioned. Note that tips 134 of each anchor member 132 remain angled slightly away from septum secundum 54 and septum primum 52 even after anchor members 132 are flattened to minimize trauma to septum secundum 54 and septum primum 52.

FIG. 5G depicts left atrial anchor 130 with two anchor members 132 of the left atrial anchor positioned against the septum primum of the heart and the other two anchor members 132 positioned against the septum secundum of the heart. In addition to a left atrial anchor with four anchor members, other configurations permit at least one anchor member 132 to be positioned against the septum primum of the heart while at least one other anchor member is positioned against the septum secundum of the heart such that the left atrial anchor remains positioned in the left atrium. For example, the left atrial anchor may have two or three anchor members or more than four anchor members. Examples of other shapes are described below in reference to FIGS. 9-11, 12A-12C, 13A-13I and 14A-14D.

Right atrial anchor 170 can be seen in its delivery configuration rotated within catheter 250 in FIG. 5F. Right atrial anchor 170 is deployed by advancing it with respect to catheter 250 by urging right atrial anchor (RAA) advancer 270 against right atrial anchor 170. Once outside of catheter 250 as shown in FIG. 5H, right atrial anchor 170 pivots into a deployed configuration such that it extends perpendicular to, or at least at an angle with respect to catheter 250. Note that at least one anchor member 132 is in a different plane relative to another anchor member 132.

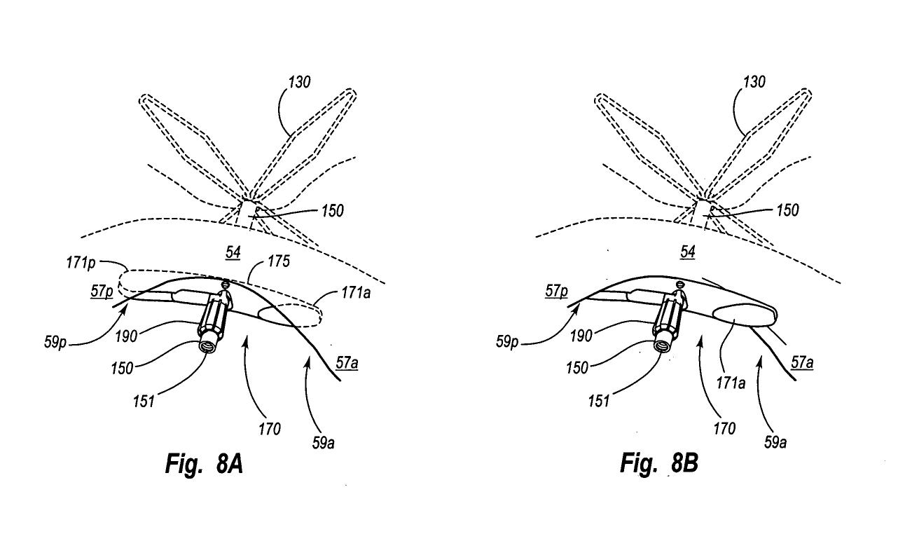

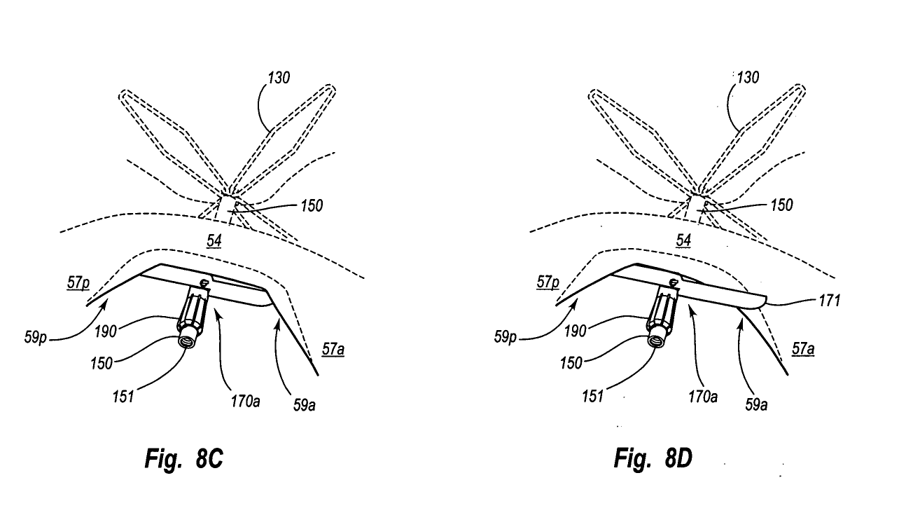

FIG. 5I shows right atrial anchor 170 being rotated clockwise. Rotation of right atrial anchor 170 is achieved by rotating stem 280 of right atrial anchor (LAA) advancer 270. Left atrial anchor 130 and right atrial anchor 170 are not brought into a locked configuration until after right atrial anchor 170 is positioned. As right atrial anchor 170 is rotated, posterior end 171p tucks under the overhang of posterior portion 57p of septum secundum 54 and in posterior pocket 59p. The posterior end of a typical septum secundum has a deeper pocket than the anterior portion of a typical septum secundum. The deeper pocket of the typical posterior end makes it easier to position an end of the right atrial anchor than under the anterior portion. Note that while FIGS. 5J-5Q depict or are described in reference to placement of the ends of right atrial anchor 170 into pocket 59a and pocket 59p at the anterior and posterior portions, closure device 100 also effectively closes a PFO when only one end of right atrial anchor 170 is positioned within pocket 59p and the other end is positioned on top of anterior portion 57a instead of in pocket 59a as discussed below with reference to FIG. 8B and FIG. 8D.

FIG. 5J depicts right atrial anchor positioned with its top surface 175 directed toward tunnel 58. FIG. 5K shows right atrial anchor 170 with its posterior end 171p partially under the overhanging posterior portion 57p of septum secundum in posterior pocket 59p and its anterior end 171a partially under the overhanging anterior portion 57a of septum secundum 54 in anterior pocket 59a.

In FIG. 5L, right atrial anchor 170 is shown after being driven toward left atrial anchor 130 on anchor connector 150 by right atrial anchor (RAA) advancer 270. Advancement of right atrial anchor 170 on anchor connector 150 enables retention pawls 199 of right atrial anchor (RAA) retainer 190 to enter retention hole 155 of anchor connector 150 so that right atrial anchor (RAA) retainer 190 is secured to anchor connector 150. Once retainer 190 locks with connector 150, right atrial anchor 170 becomes positioned further under septum secundum 54, as shown in FIG. 5M. More particularly, FIG. 5M shows right atrial anchor 170 with its posterior end 171p fully under the overhanging posterior portion 171p of septum secundum 54 in posterior pocket 59pand its anterior end 171 a fully under the overhanging anterior portion 57a of septum secundum 54 in anterior pocket 59a. With reference to FIG. 3A and FIG. 4A, note that there may be only one hole 155 while there is a plurality of retention pawls 199. This ratio and the relative widths of the hole 155 and retention pawls 199 ensures that at least one pawl 199 will be engaged in hole 155.

The sequence of steps described above with reference to FIGS. 5H-5M, indicates that the right atrial anchor 170 is first rotated clockwise into position and then right atrial anchor 170 is advanced toward left atrial anchor 130. However, these steps may also be achieved in manner which involves simultaneous clockwise rotation and advancement of right atrial anchor 170. Simultaneous rotation and advancement may involve a transition from a combination of rotation and advancement to just advancement.

FIGS. 5N-5O shows catheter 250 after removal of left atrial anchor (LAA) advancer 230. Left atrial anchor (LAA) advancer 230 can be removed after right atrial anchor 170 has been driven forward and locked with anchor connector 150 as described with reference to FIG. 5H-5M. Removal of left atrial anchor (LAA) advancer 230 is achieved by rotating stem 220 counterclockwise while maintaining tension on stem 220 and holding stem 280 secure so that threads 212 of tip 210 are no longer engaged by threads 151 of anchor connector 150. Once right atrial anchor 170 and left atrial anchor 130 have been deployed and properly positioned in the heart against the septum primum and septum secundum, as discussed above, the deployed anchors may then be detached from the remainder of the device. More particularly, after left atrial anchor (LAA) advancer 230 has been removed, then right atrial anchor advancer 270 is removed from catheter 250.

FIG. 5P-5Q depict closure device 100 in a closure position relative to PFO 50 after delivery apparatus 200 has been removed. Following deployment and positioning of the anchors, the right and left atrial anchors are left to remain in the heart on opposite sides of the PFO. The tissue at the PFO is compressed between left atrial anchor 130 and right atrial anchor 170 via anchor connector. This configuration permits closure device 100 to remain in the heart in a stable configuration and facilitate closure of the PFO.

FIGS. 6A-6F depict another embodiment of closure device which is identified as 100′ and another embodiment of delivery apparatus which is identified as 200′. The components of closure device 100′ which are different from closure device 100 include anchor connector 150′, right atrial anchor 170, and right atrial anchor (RAA) retainer 190′. The component of delivery apparatus 200′ which is different from delivery apparatus 200 includes coupler 290′ of right atrial anchor (RAA) advancer 270′. As explained below, closure device 100′ and delivery apparatus 200′ permit adjustments based on the length of the particular PFO tunnel and also permit recapture of closure device 100′ by delivery apparatus 200′.

FIGS. 6A-6B shows anchor connector 150′ having three retention holes which are identified at 155a-c. A plurality of retention holes enables retention pawls 199 of right atrial anchor (RAA) retainer 190′ to enter holes 155a-c of anchor connector 150′ until right atrial anchor 170′ is set in a desired position. As the retention pawls 199′ are moved in succession in holes 155a-c to bring right atrial anchor 170′ closer to left atrial anchor 130, the operator can identify the position of retention pawls 199′ with respect to each retention holes 155 by either feeling distinct clicks or by using instrumentation to view their position. The ability to variably set the length of the portion of anchor connector 150′ between left atrial anchor 130 and right atrial anchor 170′ is advantageous as tunnels 58 have different lengths.

FIG. 6C provides a detailed depiction of pivot collar 190′ which is another example a right atrial anchor (RAA) retainer. Pivot collar 190′ has two bands 192′ which extend around body portion 196′. Bands 192′ each have a ring portion 193′ and opposing pivot members 194′ at opposite ends of the ring portion 193′. Each pivot member 194′ extends through hole 174′ and is held in hole 174′ by ferrule 195′.

FIGS. 6D-6G and FIG. 6B show coupler 290′ and its torque feature 294′. FIG. 6D shows the portions of pivot members 194′ engaged by torque features 294′, the portion not in holes 174′ of right atrial anchor 170′. As can be seen in FIG. 6G, the space between ring portions 193′ of pivot collars 190′ and right atrial anchor 170′ is filled by coupler 290′ when torque features 294′ engage pivot members 194′. FIG. 6E shows coupler 290′ approaching pivot collar 190′. FIG. 6F shows coupler 290′ and pivot collar 190′ locked together through the engagement of torque feature 294′ and pivot member 194′.

After the anchors have been deployed on either side of the PFO, the position of the anchors may be observed via fluoroscopic, ultrasonic, or any other type of imaging available to one of skill in the art. If the anchors are in an improper or otherwise undesirable position, they may be recaptured and withdrawn or recaptured and redeployed. In the embodiment depicted in FIGS. 6A-6G, the location of the error in deployment or delivery determines where the recapture occurs. For example, if right atrial anchor 170 has been pushed through tunnel 58 and into left atrium 40 then catheter 250 is advanced distally through the PFO opening and into the left atrium so that the anchors may then be recaptured in catheter 250. Tip 210 is rotated clockwise enough turns to push retention pawls 199 out of retention holes 155 of anchor connector 150. The operator then pulls on stem 280′ of right atrial anchor (RAA) advancer 270′ while holding left atrial anchor (LAA) advancer 230. This permits right atrial anchor 170 to be pulled into catheter 250 by utilizing split tip 252 of catheter 250 to pivot right atrial anchor 170 while pulling on stem 280′ of right atrial anchor (RAA) advancer 270′. Note that each of retention pawls 199′ and holes 155 are shaped to enable retention pawls 199′ to remain in place unless lifted by tip 210 for detachment during recapture. More particularly, retention pawls 199 each have a ramp-shaped inner surface and tip 210 lifts retention pawls up so that the ramp-shaped inner surfaces may ride up the edge of holes 155 when right atrial anchor (RAA) advancer 270 is pulled. Catheter 250 recaptures left atrial anchor 130 by pulling left atrial anchor 130 into catheter 250 while split tip 252 is in the left atrium.

In contrast to having a distinct stem groove 172p and pivot groove 178 like right atrial anchor 170, right atrial anchor 170′ has a combined stem and pivot groove 178′. The combined groove 178′ is sized to permit easy access by pivot collar 190. Also, once torque feature 294′ engages pivot members 194′ and the engagement is used to pull right atrial anchor 170′ into catheter 250, space is needed within right atrial anchor 170 so that coupler 290′ can be received.

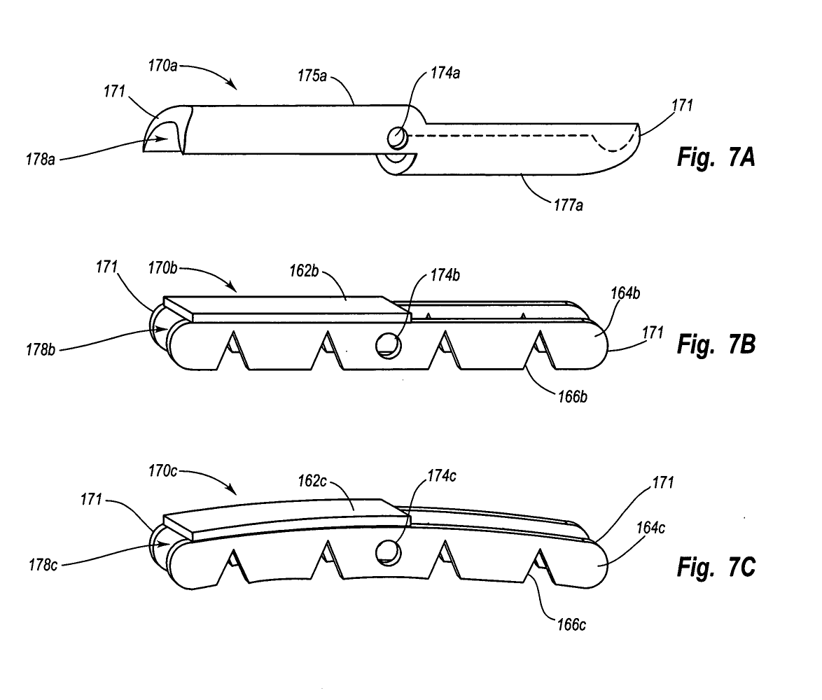

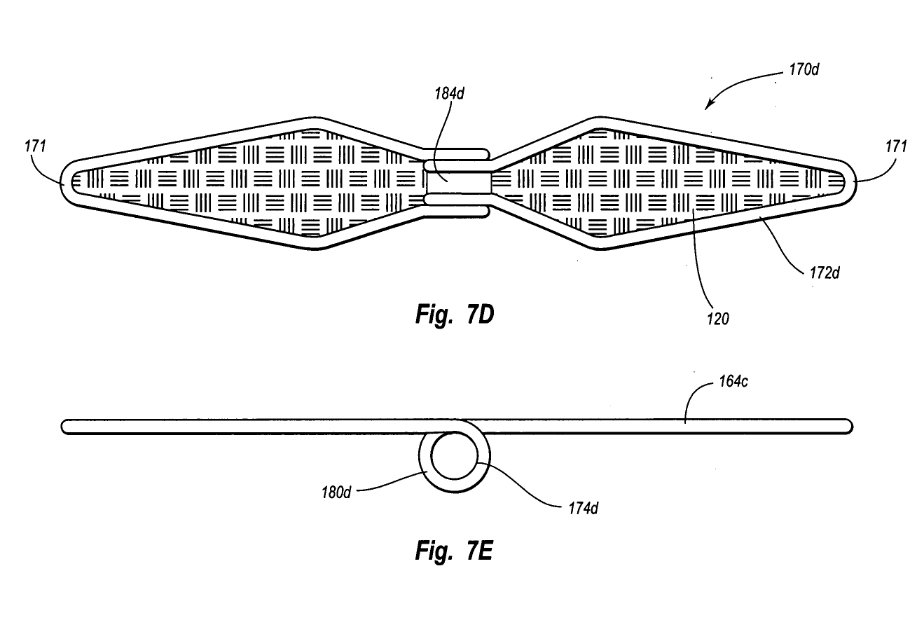

FIGS. 7A-7C depict other embodiments of right atrial anchors respectively at 170a-c. Like right atrial anchors 170 and 170′, right atrial anchor 170c has an arched shape. In contrast, right atrial anchors 170a and 170b are relatively straight. Right atrial anchors 170b and 170c have non-resorbable components 162b and 162c and resorbable components 164b and 164c. Examples of resorbable components include components formed from bioresorbable polymers and drug-eluting compositions as described above. A bio-resorbable polymer may be used to give bulk to the anchor and further to promote the formation of fibrous tissue. In such embodiments, the non-resorbable components may be used as a backbone. Although not necessary, a metal wire backbone provides for radio-opacity needed for x-ray imaging. Of course, in some embodiments the anchors and other components of the closure device may entirely comprise bio-resorbable material such that no foreign material remains in the heart after a sufficient period of time for closure of the PFO to take place. Examples of non-resorbable components include stainless steel and a super-elastic material such as nitinol. These components, like the left atrial anchor, may have any suitable cross-sectional shape. For example, left atrial anchor and the non-resorbable components of the right atrial anchor may be formed from round or flattened wire that has been formed into an appropriate shape or may be wrought from bulk material as desired.

As shown in FIG. 7A, right atrial anchor 170a has a top surface 175a and a bottom surface 177a which are both relatively straight and parallel to each other. Right atrial anchor 170a has a groove 178a which is open along its entire length except for its center.

As mentioned above and as shown in FIGS. 7B-7C, right atrial anchors 170b and 170c, respectively have non-resorbable components 162b and 162c and resorbable components 164b and 164c. In these embodiments, the resorbable component and the non-resorbable component are attached to each other. The resorbable components are segmented with notches respectively at 166b and 166c to provide enhanced flexibility. The notches facilitate flexing of the anchor into the arched configuration against the PFO.

FIGS. 7D-7E depicts another embodiment of a right atrial anchor at 170d. Right atrial anchor 170d has two opposing anchor members joined together by a loops 180 which act as flex points or regions for ends 171 to be flexed together inside a catheter when right atrial anchor 170d is in its delivery configuration. Loops 180 each define a hole 174d. Holes 174d is adapted to engage pivot members 194 or 194′ of right atrial anchor (RAA) retainer 190. An optional web 120 is shown extending within the area defined by the wire forming the opposing anchor members. Web 120 may also extend beyond the wire. A hole 184d is provided in web 120 for an anchor connector (not shown in FIGS. 7D-7E) such as anchor connector 150 or 150a.

FIGS. 8A-8D depict two different embodiments of right atrial anchors which are each positioned adjacent to a septum secundum in anatomical conformance with the septum secundum. The right atrial anchor is preferably arched with an arch which is similar to that of the septum secundum. Right atrial anchor 170 has an arched top surface 175 which is similar in shape to superior aspect 53, which is the attachment location of septum secundum 54 to septum primum 52. Right atrial anchor also has a length which permits it to be tucked under the overhang of septum secundum 54.

In addition to being rigid and having an arched configuration, the right atrial anchor can also have other shapes such as a straight configuration while being flexible so that it can conform to the arched shape of the superior aspect 53 of the septum secundum. For example, instead of right atrial anchor 170 being formed from a rigid material, it can also be formed from a more flexible material. Similarly, a flexible embodiment such as shown at 170c may be used.

FIG. 8B shows right atrial anchor 170 positioned within pocket 59p and the other end positioned on top of anterior portion 57a instead of in pocket 59a. As described above, relying on the anatomy of the posterior portion 57p of septum secundum 54 to position at least one end of right atrial anchor is an effective methodology for effectively closing a PFO. The ends of right atrial anchor are both short enough so that whichever end is positioned in pocket 59p, it conforms with the anatomy of a portion of the septum secundum.

As shown in FIGS. 8C-8D, a right atrial anchor which is rigid and straight, such as right atrial anchor 170a described above with reference to FIG. 7A, may be used. Right atrial anchor 170a has a posterior end which is short enough to fit within pocket 59p. Although, the rigidity and straight configuration of right atrial anchor 170a prevent it from curving like superior aspect 53, top surface 175a is able to abut superior aspect 53 and septum secundum 54 does not block anchor connector 150 from full access into tunnel 58. The embodiments of the right atrial anchor described above, facilitate closure of the PFO by allowing the right atrial anchor to be tucked under at least a portion of the septum secundum and against the septum primum such that the right atrial anchor can be drawn taughtly against both the septum primum and septum secundum. Healing is thereby facilitated along a greater portion of PFO tunnel 58.

At the location of a PFO, the septum primum is joined with the septum secundum at two “merger points,” as discussed above. The right atrial anchor may be shorter than the distance between these merger points to enhance the ability of the right atrial anchor to be positioned with both of its ends within pockets 59a and 59p. In other words, the right atrial anchor may extend from the point at which the septum primum is joined with the septum secundum on one end of the PFO “arch” to the point at which the septum primum is joined with the septum secundum on the other end of the PFO arch. Contact with these two merger points facilitates the right atrial anchor remaining in its proper position without being pulled through the PFO opening. Because a typical PFO has an arch that is 12-15 mm long, the right atrial anchor typically has a length of about 10 to about 30 mm although variations above and below this are contemplated in order to accommodate varying PFO anatomies. An example of a suitable right atrial anchor has a length within a range of about 15 mm to about 22 mm. An example of a suitable left atrial anchor has a length of about 15 mm to about 30 mm.

FIG. 9 depicts another embodiment of a left atrial anchor identified at 130′ which has three anchor members 132′. Left atrial anchor 130′ also has a web material or mesh 120 positioned on anchor members 132′ to further facilitate closure of PFO 50. Left atrial anchor may have any suitable number of anchor members. For example, the left atrial anchor may have just two opposing anchor members like the right atrial anchor such that both anchor members are essentially rod-shaped. Similarly, the left atrial anchor may be rod-shaped while the right atrial anchor is banana-shaped. Anchors which are rod-shaped or banana-shaped are referred to herein as elongate-shaped anchors. When both anchors have just two opposing anchor members, the right and left atrial anchors are positioned perpendicular to one another at the point of their approximation such that when they are brought together they generally form a plus (+) shape at that point. With respect to such embodiments, the right atrial anchor is typically placed in an approximately horizontal, although arched, position in the right atrium against and with respect to the PFO and the left atrial anchor is typically placed in an approximately vertical position in the left atrium against the PFO. If not configured in perpendicular orientations with respect to one another, the right and left atrial anchors will typically at least be offset from one another. In other words, the right atrial anchor will typically be positioned such that it is at an angle with respect to—i.e., not parallel to—the left atrial anchor such that are positioned in intersecting planes with respect to one another. Also, one or both anchors may have an off-center pivot point.

FIG. 10 depicts another embodiment of a closure device at 100″. Closure device 100″ has a right atrial anchor 170″ comprising a single wire looped to have opposing anchor members. Right atrial anchor 170″ is connected to left atrial anchor 130″ via an anchor connector 150″ which is a ring with either an elliptical or round shape. From the view of FIG. 10, only two anchor members of left atrial anchor 130″ are depicted. However, as understood from the juncture of the anchor members, left atrial anchor 130″, in this embodiment, has four anchor members.

FIG. 11 depicts another closure device at 100′″. Closure device 100′″ is formed from an integral material. Closure device 100′″ has an anchor connector 150 which is integral at one end with a left atrial anchor with right atrial anchor 170′″. Anchor connector 150′″ is coated with a coating which facilitates closure of PFO 50. Examples of suitable coatings include bioresorbable polymers and drug-eluting compositions. Closure device 100′″ is shaped to enable conformance with the anatomy of septum primum 52, septum secundum 54 and tunnel 58.

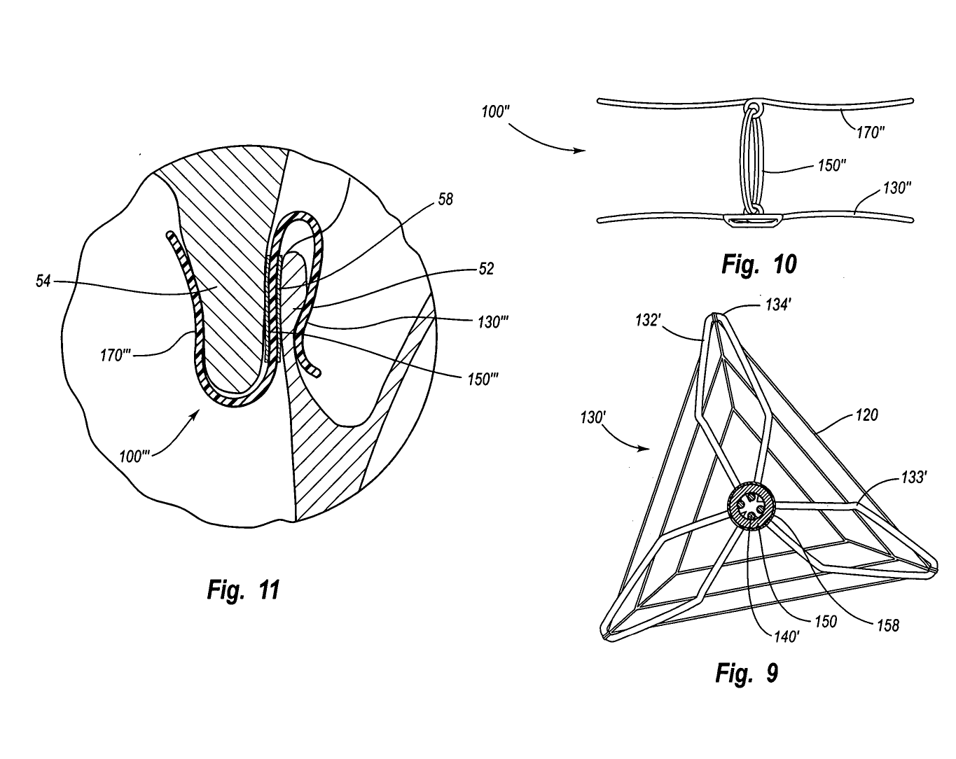

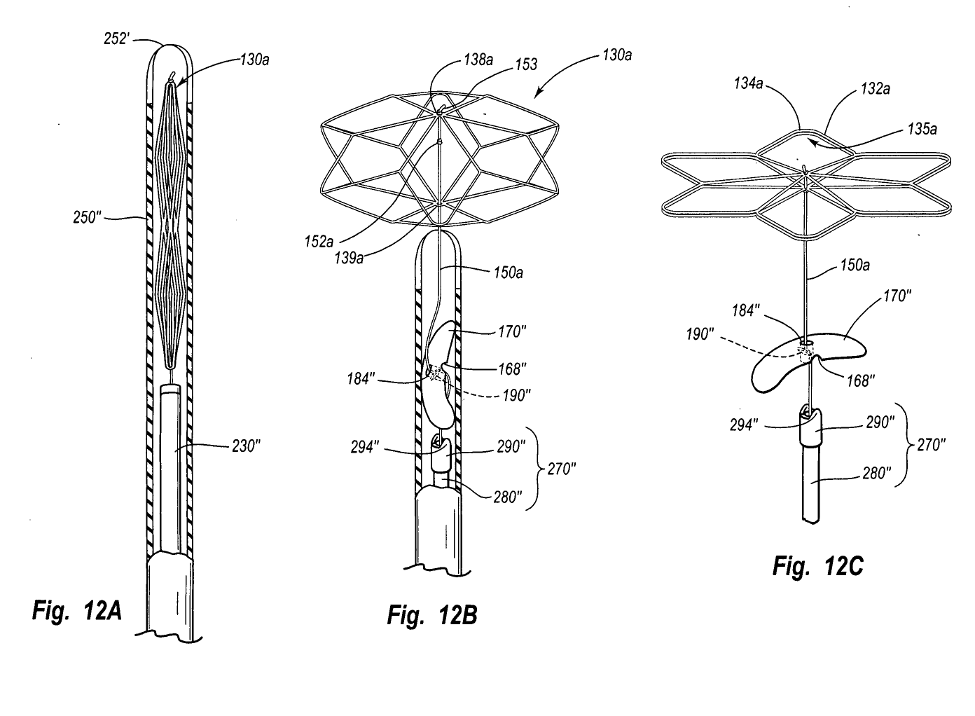

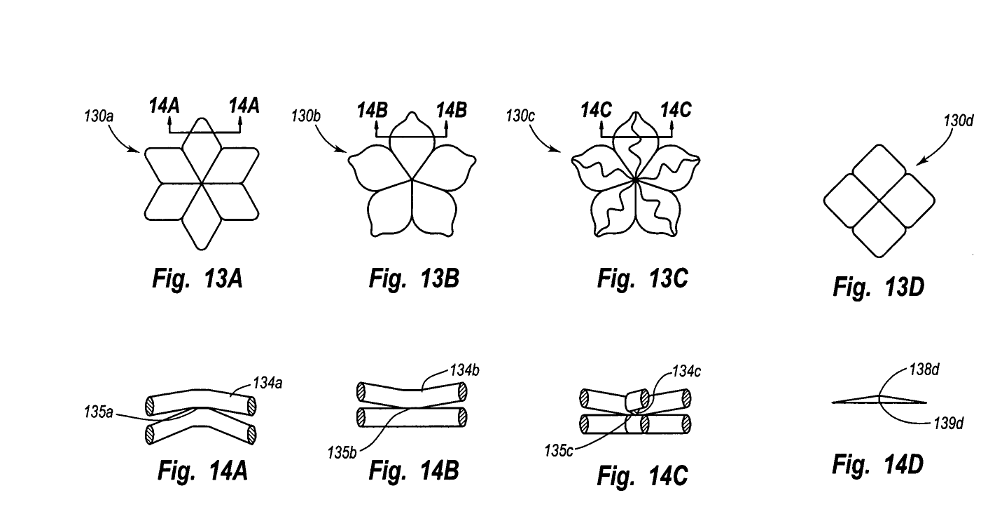

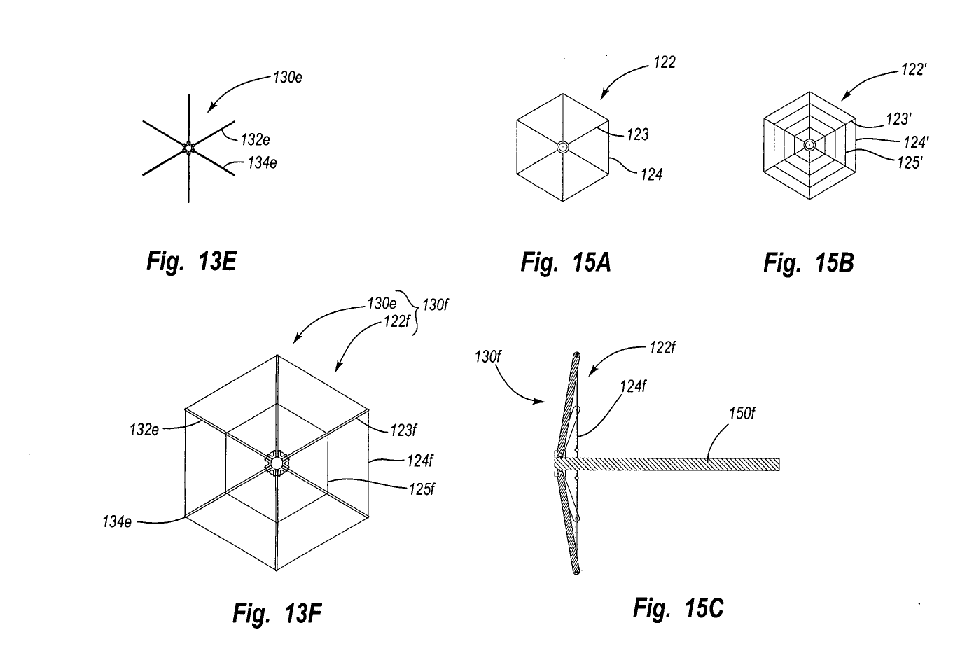

FIGS. 12A-12C depict another embodiment of a closure device 100a comprising a left atrial anchor 130a and a right atrial anchor 170″ which are connected together by an anchor connector 150a. FIGS. 12A-12C also depict 200″ another embodiment of delivery apparatus 200 having a left atrial anchor (LAA) advancer 230″ and a right atrial anchor (LAA) advancer 270″. Left atrial anchor 130a has a first set of anchor members 132a on top of a second set of anchor members 132a. The two sets are identical. The tips 134a of anchor members 132a are joined together at joints 135a. FIG. 13A provides a plan view of left atrial anchor 130a and FIG. 14A provides an enlarged cross-sectional view of joint 135a.

Left atrial anchor (LAA) advancer 230″ pushes left atrial anchor 130a out of catheter 250 and into the left atrium. FIG. 12B provides a perspective view of left atrial anchor 130a during deployment. Anchor connector 150a of closure device 100a is a thread or filament. Anchor connector 150a is tied to first center feature 138a of left atrial anchor 130a at end 153a. Anchor connector 150a has a stop 152a which is passed over by second center feature 139a of the second set of anchor members 132a as second center feature 139a is pushed towards first center feature 138a. Anchor connector 150a can be used to selectively expand or collapse left atrial anchor 130a.

FIG. 12C provides a perspective view of left atrial anchor 130a as compressed in a left atrium and right atrial anchor 170″ as positioned in the right atrium by right atrial anchor (LAA) advancer 270″. Right atrial anchor 170″ has an opening 184 through which anchor connector 150a passes. Right atrial anchor 170″ also has a right atrial anchor (RAA) retainer 190″ also referred to as a locking arm. Locking arm 190″ permits right atrial anchor 170″ to advance on anchor connector 150a toward left atrial anchor 130a. While other embodiments permit right atrial anchor 170″ to be retracted on anchor connector, locking arm 190″ does not permit right atrial anchor 170″ to be moved away from left atrial anchor 130a. Note that coupler 290″ of right atrial anchor (LAA) advancer 270″ has a torque feature 294″ for engaging torque groove 168 of right atrial anchor 170″.

Other configurations of left atrial anchor 130a having two sets of linked anchor members are shown in FIGS. 13B-13D and are identified as 130b-130d. FIGS. 14B-C provide enlarged cross-sectional views of joints 135b-c. FIG. 14D is a side view of left atrial anchor 130d being pulled slightly at its center.

FIGS. 13E-13F depict additional embodiments of left atrial anchors as identified at 130e-130f. Left atrial anchor 130e depicts an embodiment having six anchor members 132e.

FIG. 15A and FIG. 15B depict embodiments of webs respectively at 122 and 122′. Another embodiment of a web, web 122f is shown in FIG. 13F and FIG. 15C as used in combination with left atrial anchor 130e to provide left atrial anchor 130f. Web 122f comprises arm links 123f, a perimeter link 124f and an inset link 125f. Perimeter link 124f comprises link components which are either integral or separate and are attached to each end or tip 134 of each anchor member 132e. Arm links 123f and inset link 125f may also comprise link components which are either integral or separate. Web 122 shown in FIG. 15A differs from web 122f in that it does not have an inset link. Web 122′ shown in FIG. 15B differs from web 122f as web 122′ has a plurality of inset links. The inset links extending around a perimeter at certain lengths of each anchor member.

FIG. 15C depicts a plan view of left atrial anchor 130f shown in FIG. 13F with anchor connector 150f in the center of anchor 130f. The combination of webbed links on anchor members as shown in FIG. 13F permits left atrial anchors 130f to have a triangulated configuration as shown in FIG. 15C. The links may be flexible and have some tensile strength but limited compressive strength much like a string. When flexible links are used in combination with arms which are relatively rigid, the combination permits compression within a catheter in a delivery configuration and a deployed configuration which resists collapsing and being pulled into tunnel 58.

Triangulation anchors such as anchor 130f may have various configurations. For example, the links do not need to by symmetrical, integral or linked continuously on the anchor members. The webs may be formed from the same or different materials as the anchor members. For example, the anchor members may be formed from nitinol while the links are formed from resorbable polymers. Webs 122 and mesh 120 shown with reference to FIG. 9 and FIG. 7D may be used with either a left atrial anchor or a right atrial anchor. Materials may also be used as a mesh or links which have a fuzzy appearance. Triangulation atrial anchors are not shown with a web material, however, it should be understood that such an embodiment acts much like an umbrella.

Since the embodiments disclosed herein have right and left atrial anchors that are coupled to one another—i.e., they are integral, attached, or otherwise connected with one another—once the anchors have each been deployed, they will remain in place on either side of the PFO opening.

Right atrial anchor and left atrial anchor can be coupled together by any available structure or in any available manner. For example, the respective anchors may be considered “coupled” if they are integral, attached, or otherwise connected with one another. The atrial anchor may be shaped to provide a torsion-spring-like flexural pivot that minimizes strain in the anchor material as it is deformed between the delivery configuration and the deployed configuration and vice versa. Note that while anchor connectors 150, 150′ and 150a are shown as the structure for coupling the right and left atrial anchors, some embodiments of the invention don't have a connector at all. For example, portions of the anchors may extend into or through tunnel 58 to join the anchors together. Also, the anchors could be welded, glued, or integrally connected. Moreover, a variety of other suitable structures or other arrangements could be used to connect the anchors, such as a cable, filament, chain, clip, clamp, band, or any other manner of connection available to those of skill in the art.

The left atrial anchors disclosed herein are examples of left atrial anchor means for anchoring a closure device in the left atrium of a heart. The right anchor disclosed herein are examples of right atrial anchor means for anchoring a closure device in the right atrium of a heart. Mesh disclosed herein is an example of means for increasing the surface area of the atrial anchor. Webs disclosed herein are means for preventing an atrial anchor from extending beyond the deployed configuration. The anchor connectors disclosed herein are examples of means for connecting the right atrial anchor means and the left atrial anchor means. Coatings and components of a closure device formed from a bioresorbable polymer, a drug eluting composition, a protein, a growth factor or a combination thereof.etc. are examples of means for enhancing mechanical closure of a PFO. Left atrial anchor retainers disclosed herein are examples of left atrial anchor retainer means for retaining the left atrial anchor on the anchor connector. Right atrial anchor retainers herein are examples of right atrial anchor retainer means for retaining the right atrial anchor on the anchor connector. Left atrial anchor (LM) advancers disclosed herein are examples of means for controlling the position of the left atrial anchor. Right atrial anchor (LAA) advancers disclosed herein are examples of means for controlling the position of the right atrial anchor. The catheters disclosed herein are examples of means for positioning the closure device. The closure devices disclosed herein are examples of means for closing a PFO.

All publications, including but not limited to patents and patent applications, cited in this specification are herein incorporated by reference as if each individual publication were specifically and individually indicated to be incorporated by reference herein as though fully set forth.

The above description fully discloses the invention including preferred embodiments thereof. Without further elaboration, it is believed that one skilled in the art can use the preceding description to utilize the invention to its fullest extent.

It will be apparent to those having skill in the art that changes may be made to the details of the above-described embodiments without departing from the underlying principles of the invention. Embodiments of the invention in which an exclusive property or privilege is claimed are defined as follows. Note that elements recited in means-plus-function format are intended to be construed in accordance with 35 U.S.C. § 112 ¶6.

1. A method for facilitating closure of a patent foramen ovale (PFO), comprising the steps of:
obtaining a closure device comprising a left atrial anchor coupled with a right atrial anchor;
deploying the left atrial anchor of the device in the left atrium of a heart and positioning the left atrial anchor proximate to the PFO such that one end of the left atrial anchor is positioned against the septum primum of the heart and the other end of the left atrial anchor is positioned against the septum secundum of the heart; and
deploying the right atrial anchor in the right atrium of the heart and positioning the right atrial anchor proximate to the opposite side of the PFO such that at least a portion of the right atrial anchor is tucked under the septum secundum and against the septum primum in conformance with the anatomy of the PFO to enable the left atrial anchor and right atrial anchor to be respectively held in the left atrium and the right atrium against the septum primum and septum secundum.

2. The method of claim 1, wherein the closure device further comprises an anchor connector, wherein the anchor connector is connected to the left atrial anchor at one end and connected to the right atrial anchor at the other end such that the angle of the right atrial anchor in the right atrium relative to the anchor connector and the angle of the left atrium anchor in the left atrium relative to the anchor connector permit the anchor connector to conform to the anatomy of the tunnel of the PFO.

3. The method of claim 1, wherein the closure device further comprises an anchor connector, wherein the anchor connector is connected to the left atrial anchor in a configuration which permits the left atrial anchor to pivot in any plane, and wherein the anchor connector is connected to the right atrial anchor in a configuration which permits the right atrial anchor to pivot in only one plane.

4. The method of claim 1, wherein the right atrial anchor is flexible.

5. The method of claim 1, wherein the right atrial anchor is arched.

6. The method of claim 1, wherein the right atrial anchor has a contact surface which is relatively straight.

7. The method of claim 1, wherein the right atrial anchor is integral.

8. The method of claim 1, wherein the right atrial anchor has two opposing anchor members joined together at a flexible region.

9. The method of claim 1, wherein at least one of the right atrial anchor and the left atrial anchor is coated with at least one of a bioresorbable polymer, a drug eluting composition, a protein, a growth factor and a combination thereof.

10. The method of claim 1, wherein at least one of the right atrial anchor and the left atrial anchor comprises at least one of a bioresorbable polymer, a drug eluting composition, a protein, a growth factor and a combination thereof.

11. The method of claim 1, wherein the left atrial anchor comprises at least three anchor members.

12. The method of claim 1, wherein deploying the right atrial anchor involves rotating the right atrial anchor clockwise until one of its ends is tucked under a portion of the septum secundum.

13. The method of claim 1, wherein the right atrial anchor has two opposing anchor members and an axis extending through the anchor members and wherein the axis of the deployed right atrial anchor intersects the plane of the left atrial anchor as deployed against the heart.

14. A method for facilitating closure of a patent foramen ovale (PFO), comprising the steps of:
obtaining a device comprising a left atrial anchor coupled with a right atrial anchor, wherein the right atrial anchor has only two opposing anchor members;
deploying the left atrial anchor of the device in the left atrium of a heart and positioning the left atrial anchor proximate to the PFO such that such that a portion of the left atrial anchor is positioned against the septum primum of the heart and another such that a portion of the left atrial anchor is positioned against the septum secundum of the heart; and
deploying the right atrial anchor in the right atrium of the heart and positioning the right atrial anchor proximate to the opposite side of the PFO such that at least a portion of the right atrial anchor conforms with the anatomy of the PFO in an arched configuration with at least a portion of the right atrial anchor tucked under the septum secundum and against the septum primum such that the left atrial anchor and right atrial anchor are respectively held in the left atrium and the right atrium against the septum primum and septum secundum.

15. The method of claim 14, wherein deploying the right atrial anchor enables the right atrial anchor to extend at least as long as a portion of the arch in the inferior aspect of the septum secundum between the merger points of the PFO.

16. A method for facilitating closure of a patent foramen ovale (PFO), comprising the steps of:
introducing a catheter containing a device comprising a left atrial anchor coupled with a right atrial anchor through the PFO and into the left atrium of a patient's heart;
deploying the left atrial anchor in the left atrium from the catheter;
positioning the left atrial anchor proximate to the PFO such that a portion of the left atrial anchor is positioned against the septum primum of the heart and another portion of the left atrial anchor is positioned against the septum secundum of the heart;
deploying the right atrial anchor in the right atrium of the heart from the catheter; and
positioning the right atrial anchor proximate to the opposite side of the PFO such at least a portion of the right atrial anchor generally follows the arch formed by the inferior aspect of the septum secundum along the periphery of the PFO.

17. The method of claim 16, wherein the right atrial anchor has at least one end extending toward one of the merger points at which the septum secundum and septum primum come together at the edges of the PFO.

18. The method of claim 16, further comprising the step of recapturing the right and left atrial anchors in the catheter.

19. A device for facilitating closure of a patent foramen ovale (PFO), comprising:
a left atrial anchor adapted to be positioned at the PFO in the left atrium of the heart in a deployed configuration; and
a right atrial anchor,
wherein the right atrial anchor is adapted to fit within a catheter in a delivery configuration and adapted to be positioned at the PFO in the right atrium of the heart in a deployed configuration,
wherein the right atrial anchor has only two opposing anchor members; and
wherein the right atrial anchor has a length and shape which permit at least a portion of the right atrial anchor to be tucked under the septum secundum and against the septum primum in conformance with the anatomy of the PFO; and
an anchor connector, wherein the anchor connector is connected to the left atrial anchor at one end and connected to the right atrial anchor at the other end such that the angle of the right atrial anchor in the right atrium relative to the anchor connector and the angle of the left atrium anchor in the left atrium relative to the anchor connector permit the anchor connector to conform to the anatomy of the tunnel.

20. The device of claim 19, wherein the right atrial anchor is rigid.

21. The device of claim 19, wherein the right atrial anchor is flexible.

22. The device of claim 19, wherein the right atrial anchor is arched.

23. The device of claim 19, wherein the right atrial anchor has a contact surface which is relatively straight.

24. The device of claim 19, wherein the right atrial anchor is integral.

25. The device of claim 19, wherein the two opposing anchor members of the right atrial anchor are joined together at a flexible region.

26. The device of claim 19, wherein at least one of the right atrial anchor and the left atrial anchor is coated with at least one of a bioresorbable polymer, a drug eluting composition, a protein, a growth factor and a combination thereof.

27. The device of claim 19, wherein at least one of the right atrial anchor and the left atrial anchor comprises at least one of a bioresorbable polymer, a drug eluting composition, a protein, a growth factor and a combination thereof.

28. The device of claim 19, wherein the left atrial anchor comprises at least three anchor members.

29. The device of claim 19, wherein the left atrial anchor comprises a plurality of anchor members and at least one anchor member is in a different plane relative to another anchor member.

30. A device for facilitating closure of a patent foramen ovale (PFO), comprising:
a left atrial anchor adapted to be positioned at the PFO in the left atrium of the heart in a deployed configuration,
wherein the left atrial anchor is adapted to pivot in any plane; and
a right atrial anchor coupled with the left atrial anchor, wherein the right atrial anchor is adapted to fit within a catheter in a delivery configuration and adapted to be positioned at the PFO in the right atrium of the heart in a deployed configuration,
wherein the right atrial anchor has two opposing anchor members, and
wherein the right atrial anchor is adapted to pivot in only one plane.

31. A device for facilitating closure of a patent foramen ovale (PFO), comprising:
a left atrial anchor adapted to be positioned at the PFO in the left atrium of the heart; and
a right atrial anchor coupled with the left atrial anchor, wherein the right atrial anchor is adapted to fit within a catheter in a delivery configuration and adapted to be positioned at the PFO in the right atrium of the heart in a deployed configuration,
wherein the right atrial anchor has two opposing anchor members, and,
wherein the right atrial anchor has a length and shape which permit at least a portion of the right atrial anchor to be tucked under the septum secundum and against the septum primum in conformance with the anatomy of the PFO.

32. A device for facilitating closure of a patent foramen ovale (PFO), comprising:
a left atrial anchor adapted to fit within a catheter in a delivery configuration and adapted to be positioned at the PFO in the left atrium of the heart in a deployed configuration; and
a right atrial anchor, wherein the right atrial anchor is adapted to fit within a catheter in a delivery configuration and adapted to be positioned at the PFO in the right atrium of the heart in a deployed configuration, wherein the, right atrial anchor has two opposing anchor members; and
an anchor connector, wherein the anchor connector is connected to the left atrial anchor in a configuration which permits the left atrial anchor to pivot in any plane, and wherein the anchor connector is connected to the right atrial anchor in a configuration which permits the right atrial anchor to pivot in only one plane.

33. The device of claim 32, wherein at least one of the right atrial anchor, the left atrial anchor and the anchor connector is coated with at least one of a bioresorbable polymer, a drug eluting composition, a protein, a growth factor and a combination thereof.

34. The device of claim 32, wherein at least one of the right atrial anchor, the left atrial anchor and the anchor connector comprises at least one of a bioresorbable polymer, a drug eluting composition, a protein, a growth factor and a combination thereof.

35. A device for facilitating closure of a patent foramen ovale (PFO), comprising:
a left atrial anchor adapted to fit within a catheter in a delivery configuration and adapted to be positioned at the PFO in the left atrium of the heart in a deployed configuration, the left atrial anchor comprising a plurality of anchor members radially extending from a center and a web, wherein the web has link components which link the anchor members in a manner which permits the anchor members to pivot in a limited angle between the delivery configuration to the deployed configuration while preventing further pivoting beyond the deployed configuration; and
a right atrial anchor coupled with the left atrial anchor.

36. The device of claim 35, wherein the anchor members have ends and the link components are attached to each end of each anchor member to form a perimeter link.

37. The device of claim 35, further comprising an anchor connector, wherein the anchor connector is connected to the left atrial anchor and the right atrial anchor.

38. A device for facilitating closure of a patent foramen ovale (PFO), comprising:
a left atrial anchor adapted to be positioned at the PFO in the left atrium of the heart; and
a right atrial anchor adapted to be positioned at the PFO in the right atrium of the heart;
an anchor connector, wherein the anchor connector is integrally connected to the left atrial anchor and integrally connected to the right atrial anchor; and
a coating on at least a portion of the anchor connector which facilitates closure of a patent foramen ovale.

39. The device of claim 38, wherein the coating is at least one of a bioresorbable polymer, a drug eluting composition, a protein, a growth factor and a combination thereof.

40. The device of claim 38, wherein at least one of the right atrial anchor, the left atrial anchor and the anchor connector comprises at least one of a bioresorbable polymer, a drug eluting composition, a protein, a growth factor and a combination thereof.

41. An apparatus for facilitating closure of a patent foramen ovale (PFO), comprising:
a left atrial anchor adapted to fit within a catheter in a delivery configuration and adapted to be positioned at the PFO in the left atrium of the heart in a deployed configuration; and
a right atrial anchor coupled with the left atrial anchor that is adapted to fit within a catheter in a delivery configuration and adapted to be positioned at the PFO in the right atrium of the heart in a deployed configuration, wherein the right atrial anchor is shaped and adapted to be placed along the arch formed by the inferior aspect of the septum secundum along the periphery of the PFO in the right atrium of the heart, wherein the right atrial anchor has a length which enables the right atrial anchor to extend along the arch between the merger points at which the septum secundum and septum primum come together at the edges of the PFO.

42. A system for facilitating closure of a patent foramen ovale (PFO), comprising:
a device comprising:
a left atrial anchor adapted to be fit within the catheter and to be positioned at the PFO in the left atrium of the heart in a deployed configuration; and
a right atrial anchor coupled with the left atrial anchor that is adapted to fit within a catheter in a delivery configuration and adapted to be positioned at the PFO in the right atrium of the heart in a deployed configuration, wherein the right atrial anchor is shaped and sized to permit at least a portion of the right atrial anchor along the arch formed by the inferior aspect of the septum secundum along the periphery of the PFO in the right atrium of the heart;
and a delivery apparatus comprising:
a catheter,
means for controlling the position of the left atrial anchor; and
means for controlling the position of the right atrial anchor.

43. The system of claim 42, wherein the means for controlling the position of the left atrial anchor and the means for controlling the position of the right atrial anchor also serve to allow the device to be recaptured in the catheter.
